SEQUENCE LISTING (7 sequences)

SEQ ID NO: 1 - protein, 906 aa, Homo sapiens
MAGNVKKSSGAGGGTGSGGSGSGGLIGLMKDAFQPHHHHHHHLSPHPPGTVDKKMVEKCWKLMDKVVRLCQNPKLALKNSPPYILDLLPDTYQHLRTILSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTGLCEPTPQDHIKVTQEQYELYCEMGSTFQLCKICAENDKDVKIEPCGHLMCTSCLTSWQESEGQGCPFCRCEIKGTEPIVVDPFDPRGSGSLLRQGAEGAPSPNYDDDDDERADDTLFMMKELAGAKVERPPSPFSMAPQASLPPVPPRLDLLPQRVCVPSSASALGTASKAASGSLHKDKPLPVPPTLRDLPPPPPPDRPYSVGAESRPQRRPLPCTPGDCPSRDKLPPVPSSRLGDSWLPRPIPKVPVSAPSSSDPWTGRELTNRHSLPFSLPSQMEPRPDVPRLGSTFSLDTSMSMNSSPLVGPECDHPKIKPSSSANAIYSLAARPLPVPKLPPGEQCEGEEDTEYMTPSSRPLRPLDTSQSSRACDCDQQIDSCTYEAMYNIQSQAPSITESSTFGEGNLAAAHANTGPEESENEDDGYDVPKPPVPAVLARRTLSDISNASSSFGWLSLDGDPTTNVTEGSQVPERPPKPFPRRINSERKAGSCQQGSGPAASAATASPQLSSEIENLMSQGYSYQDIQKALVIAQNNIEMAKNILREFVSISSPAHVAT

SEQ ID NO: 2 - RNA, 19 nt, Artificial
  iRNA
gggaaggcuucuauuuguu

SEQ ID NO: 3 - RNA, 19 nt, Artificial
  iRNA
cuguccaucuagagacaaa

SEQ ID NO: 4 - DNA, 30 bp, Artificial
  RT-PCR primer
aaaaaggggaaagaggagtgcgcttgtcaa

SEQ ID NO: 5 - DNA, 30 bp, Artificial
  RT-PCR primer
attatatgtcttttggcctcctctctactg

SEQ ID NO: 6 - DNA, 26 bp, Artificial
  primer
atggacaaggttggtgcggttgtggt

SEQ ID NO: 7 - DNA, 24 bp, Artificial
  primer
gaagaggctgatagtctgcttagt

(19) United States
(12) Patent Application Publication
(10) Pub. No.: US 2012/0076788 A1
(43) Pub. Date: Mar. 29, 2012

(54) C-CBL AND ANTAGONISTS THEREOF FOR THE TREATMENT AND DIAGNOSIS OF CANCER

(75) Inventors: Daniel Regnier, Vienne (FR); Serge Manie, Thie (FR); Sadok Yakoub, Saint Priest (FR); Eric Tabone, St Cyr Sur Le Rhone (FR)

(73) Assignees: Centre National De La Recherche Scientifique (C.N.R.S.), Paris (FR); Centre Leon-Berard, Lyon Cedex 08 (FR); Universite Claude Bernard Lyon 1, Vileurbanne (FR)

(21) Appl. No.: 13/148,870

(22) PCT Filed: Feb. 10, 2010
(86) PCT No.: PCT/EP2010/051644

§ 371 Date: Dec. 12, 2011

Related U.S. Application Data
(63) Continuation of application No. 12/369,309, filed on Feb. 11, 2009.

(51) Int. Cl. A61K 39/395; A61K 31/7088; G01N 33/573; A61K 31/711; G01N 33/574; A61K 31/167; A61P 35/00; A61K 31/7105; C12Q 1/68

(52) U.S. Cl. 424/138.1; 514/44 A; 514/44 R; 435/7.4; 435/6.13; 435/6.12; 514/629

(57) ABSTRACT

The present invention relates to the treatment of cancer. More specifically, the present invention relates to the use of c-cbl as a marker for the diagnosis and/or prognosis of cancer, and to the use of a c-cbl antagonist for the treatment of a cancer associated with resistance to apoptosis.

### FIELD OF THE INVENTION

The present invention relates to the treatment of cancer. More specifically, the present invention relates to the use of c-cbl as a marker for the diagnosis and/or prognosis of cancer, and to the use of a c-cbl antagonist for the treatment of a cancer associated with resistance to apoptosis.

### BACKGROUND OF THE INVENTION

Cancer and Apoptosis

Defective apoptosis (programmed cell death) represents a major causative factor in the development and progression of cancer. The ability of tumor cells to evade engagement of apoptosis can play a significant role in their resistance to conventional therapeutic regimens. Cancer cells typically possess a number of mutations that have allowed them to ignore normal cellular signals regulating their growth and become more proliferative than normal. In the case of cancer associated with resistance of apoptosis, the development of tumors arises as a consequence both of dysregulated proliferation and of a suppression of apoptosis. Each of these primary defects provides an opportunity for clinical intervention. However, many of the current chemotherapeutics designed to perturb proliferation do so in such a crude manner that the resulting damage to normal cells limits their clinical efficacy. In addition, most of the cancer treatments rely on damaging the cells with radiation or chemicals, and often lead to selection of cells that are resistant to this type of attack. Finally, many of the conventional drugs are not effective on cancer cells that are resistant to apoptosis. Several studies have shown that most, if not all, chemotherapeutic agents exert their anticancer activity by inducing apoptosis; therefore, resistance to apoptosis may be a major factor limiting the effectiveness of anticancer therapy.

Prostate cancer is among the most frequently diagnosed cancer in men in Western countries and accounts for 15.3% of all cancers in men. It is the second or third leading cause of cancer death. Its incidence is increasing and is predicted to be the most common male malignancy by 15 years.

The gravity of this cancer then comes from its unavoidable progression after a few years of evolution to androgen unresponsiveness and numerous studies have focused on understanding the molecular events that lead to androgen-refractory prostate cancer. If it is unclear why prostate tumors becomes androgen independent, the molecular events that govern the neoplastic transformation at the very beginning in elderly men is also poorly understood.

However, alteration of programmed cell death represents the main explanation for gradual accumulation of prostate cancer cells in human. This alteration is indeed obvious in androgen-insensitive prostate tumors, which are resistant to several chemotherapeutic drugs and to apoptosis initiation, even if the apoptotic machinery is still in place. Androgen unresponsiveness could be either due to unregulated cell proliferation and/or over-expression of anti-apoptotic factors. It is reported that Inhibitor of Apoptosis (IAP) family proteins are involved in apoptosis resistance in some cancers, particularly in prostate. These inhibitors act at the very end of the apoptotic cascade, at the level of initiator and effector caspases. XIAP for instance has been shown to have an inhibitory effect on cell death induced by a variety of apoptotic stimuli leading to chemotherapy resistance. But very interestingly, it has also been reported that increased IAP expression was observed as soon as carcinoma in situ (PINs), suggesting that this apoptosis deregulation occurs early in the pathogenesis of prostate cancer and did not correlate with Gleason grade or Prostate-Specific Antigen (PSA) level. Thus, while the commonly used PSA assay gives important diagnostic indications, it is however reliable neither for diagnosis nor for monitoring progression. In particular, the PSA assay leads to false positives, and sometimes to false negatives. In addition, there is no true correlation with the seriousness of the disease since the PSA level may be high even in case of non aggressive prostate cancer. Finally, there is no curative treatment for prostate cancer when the tumor has crossed the androgen-dependent phase, which occurs after approximately 2 years of tumor progression.

Thus there is a need for a reliable diagnostic tool, and for treatments allowing restoring apoptosis in cancer cells that have become resistant to apoptosis.

### The c-cbl Proto-Oncogene

The c-cbl proto-oncogene acts as a negative regulator of several receptor protein tyrosine kinase signaling pathways, and as an adaptor protein in tyrosine phosphorylation-dependent signaling. More specifically, c-cbl has an E3 ligase function, and its role as a multidomain adaptor protein is well documented. It has been known for many years that c-cbl acts as a negative regulator of a certain number of growth factor receptors (RTKs) such as e.g. EGF-R, PDGF-R and CSF-1.

Wild-type c-cbl (also referred to as p120^{cbl}) is not oncogenic. However, several mutants of c-cbl have been shown to be oncogenic (Hamilton et al. 2001 J. Biol. Chem. 276:9028-9037; Sinha et al. 2001 Exp Hematol. 29:746-55; Thien et al. 2005 EMBO J. 24:3807-3819). These mutations hardly have an effect on cell proliferation. The expression of the CblΔY371 oncogenic mutant was then shown to suppress apoptosis in mice (Hamilton et al. 2001 J. Biol. Chem. 276:9028-9037).

Since (i) c-cbl negatively modulates RTKs by inducing their degradation; and (ii) mutations in c-cbl suppress or deplete apoptosis, it is currently believed in the art that c-cbl inactivation is responsible of resistance to apoptosis and development of cancers. Consequently, it is currently believed in the art that activation and/or administration of c-cbl should be beneficial for the treatment of cancer.

For example, WO/1999/067380 teaches the administration of an expression vector encoding c-cbl in order to treat or to prevent cancer. El Chami et al. (2005; J Cell Biol. 171:651-61) further teaches that c-cbl expression is mandatory to activate androgen-dependent apoptosis in testicular germ cells.

Other negative regulators of RTKs include Sprouty 2. Sprouty 2 acts as an inhibitor of FGF-R and of EGF-R and is involved in the regulation of the RTK RAS/MAPK pathway. Sprouty 2 was shown to negatively regulate the E3-ubiquitin ligase function of c-Cbl. Sprouty 2 binds to the c-cbl domain that is required for binding of c-cbl to E2 ubiquitin, thus preventing RTK degradation. Based on this fact and on the fact that increased Sprouty 2 expression is found in some cancers, WO/2006/113579 teaches that Sprouty should be inhibited in order treat cancer. As in WO/1999/067380, the idea underlying this teaching is that it is advisable to activate c-Cbl in order to increase the negative regulation exerted by c-cbl on RTKs.

A recent publication by Edwin and Patel (2008; J. Biol. Chem. 283:3181-3190) reinforces this hypothesis. This publication teaches that in the SW13 cancer cell line, Sprouty 2 inhibition antagonizes the protection against apoptosis provided by the serum added to the culture medium and decreases the phosphorylation of Akt and of Erk1/2. According to this publication, the negative regulation by Sprouty 2 involves c-Cbl since Sprouty 2 has no effect on the resistance to apoptosis induced by the presence of serum when c-Cbl is knocked out.

Another recent publication by Khan et al. (FASEB J. 2008; 22:910-7) teaches that oxidative stress induced either by cigarette smoke or by H₂O₂ causes aberrant phosphorylation of EGF-R, thereby abrogating binding of c-cbl to EGF-R. As a consequence, EGF-R is not only activated but also stabilized. Again, idea underlying this teaching is that oxidative stress leads to inactivation of c-cbl, thereby leading to enhanced cell survival. In other words, c-cbl is suggested to be a pro-apoptotic regulator that is inactivated under oxidative stress conditions as cancer or cigarette smoke.

### DESCRIPTION OF THE INVENTION

The inventors of the present patent application have surprisingly found that the c-cbl proto-oncogene acts in fact as a negative and not as a positive regulator of apoptosis.

It has been found that c-cbl is overexpressed in malignant human prostate tumors. C-Cbl is a marker for prostate cancer, and its expression level is positively correlated with the seriousness of prostate cancer.

The expression of c-cbl was studied in human prostate tumors at the androgen-dependent stage. C-cbl expression was greatly increased in these tissues (up to 7 times higher than in the surrounding healthy tissue). c-cbl expression level was proportional to the seriousness of the cancer, as assessed according to the Gleason score. C-cbl is therefore a prognostic marker for prostate cancer.

Additional analyses by immunohistochemistry were carried out on benign prostatic hyperplasia (BPH). C-cbl is expressed in epithelial cells of BPHs, but at a much lower level than in serious prostate cancers.

Further analyses by immunochemistry allowed demonstrating that c-cbl is expressed in other cancers than prostate cancer. More specifically, it is expressed in lung cancer, breast cancer, ovary cancer, brain cancer, colon cancer, colorectal cancer, thyroid cancer, testicular cancer, lymphoma and melanoma as well.

In addition, analyses of mouse embryonic fibroblasts (MEFs) originating from c-cbl knockout animals (referred to as MEF KO) or wild-type animals (referred to as MEF WT) confirmed these results and clearly showed the anti-apoptotic role of the wild-type form of c-cbl (referred to as p120^{cbl}). More specifically, c-cbl protects MEFs from apoptosis induced by an oxidative stress caused by H₂O₂. Conversely, c-cbl does not protect MEFs from apoptosis induced by etoposide. Therefore, oxidative stress is believed to cause an increase in c-cbl expression levels, which in turn protects from apoptosis (i.e. apoptosis resistance). Cancer cells being under oxidative stress conditions, these results lead to the conclusion that the resistance to apoptosis of tumor cells is due, at least in part, to the increased c-cbl expression that is caused by oxidative stress.

Finally, further experiments confirming the anti-apoptotic role of p120^{cbl} have been carried out. These experiments involved studying the expression of inhibitors of apoptosis (IAPs). It was shown that the negative regulation of apoptosis exerted by c-cbl in MEFs also involves the regulation of IAPs. It has been shown in vitro in MEFs that the absence of p120^{cbl} led to a significant decrease of expression of the XIAP protein, and to a lesser extent, to a decreased expression of the c-IAP1 and c-IAP2 proteins. Therefore, increased c-cbl activity in tumor cells is believed to cause an increased expression of inhibitors of apoptosis (IAPs). Once again, these results lead to the conclusion that the resistance to apoptosis of tumor cells is due, at least in part, to the increased c-cbl expression, which in turn leads to a decreased expression of IAPs.

In summary, the inventors report a new antiapoptotic effect of c-Cbl both in vivo and in primary KO mouse-embryonic-fibroblasts (MEFs). KO mice have a slight over-regulation of the mitochondrial spontaneous or androgen-sensitive apoptosis pathway in prostate epithelial-cells, as do KO MEFs subjected to p53 dependent-apoptosis. It was further showed that KO MEFs support a drastic apoptosis in oxidative stress conditions, and that a robust c-cbl over-expression in malignant tumours of diverse origin should be linked to the aggressiveness of the disease. Finally, it was shown that c-Cbl disruption plays a role in the decrease of oxidative stress. More specifically, c-cbl was found to contribute to the apoptose-resistance of tumor cells by increasing their high production of reactive oxygen species (ROS).

Thus, on the one hand, the expression level of c-cbl enables diagnosing and/or evaluating of the seriousness of tumors. On the other hand, the therapeutic targeting of c-cbl should contribute to reducing the expression of inhibitors of apoptosis (IAPs) in tumor cells, and should thus contribute to reducing or abolishing the resistance to apoptosis of tumor cells. In addition, targeting of c-cbl should also contribute to reducing the production of reactive oxygen species (ROS), which are believed to be a major cause of the resistance to apoptosis observed for tumor cells. Targeting of c-cbl should thus abolishing the resistance to apoptosis of tumor cells through two different mechanisms. Therefore, the present invention relates to the use of c-cbl as a marker for the diagnosis and/or prognosis of cancer, and to the use of a c-cbl antagonist for the treatment of a cancer associated with resistance to apoptosis and more generally for the treatment of any disease linked with apoptosis.

### Therapeutic Use of c-cbl Antagonists

A first aspect of the invention is method of treating or preventing a cancer, in particular a cancer associated with resistance to apoptosis, comprising the step of administering an effective amount of a c-cbl antagonist to an individual in need thereof. This aspect also relates to a c-cbl antagonist for use in treatment or prevention of a cancer, in particular of a cancer associated with resistance to apoptosis.

As used herein, the term “cancer” refers to any type of malignant (i.e. non benign) tumor. The tumor may correspond to a solid malignant tumor, which includes e.g. carcinomas, adenocarcinomas, sarcomas, malignant melanomas, mesotheliomas, blastomas, or to a blood cancer such as leukemias, lymphomas and myelomas. The carcinoma or adenocarcinoma may for example correspond to a bladder, a colon, a kidney, an ovary, a prostate, a lung, an uterus, a breast or a prostate carcinoma or adenocarcinoma. The blastoma may for example correspond to a neuroblastoma, a glioblastoma or a retinoblastoma. The cancer is preferably selected from the group consisting of prostate cancer (e.g. prostate adenocarcinoma), lung cancer (e.g. squamous cellular carcinoma), breast cancer (e.g. infiltrated ductal carcinoma), ovary cancer (e.g. serous papillary carcinoma), uterus cancer (squamous cellular carcinoma), brain cancer (e.g. astrocytoma), colon cancer (e.g. colon adenocarcinoma), colorectal cancer, rectal cancer (e.g. rectal adenocarcinoma), cancer of the striated muscle (e.g. rhabdomyosarcoma), thyroid cancer, testicular cancer, lymphoma and melanoma. In a most preferred embodiment, the cancer is selected from the group consisting of lung cancer, prostate cancer, ovary cancer, uterus cancer, brain cancer, colon cancer, colorectal cancer, rectal cancer and cancer of the striated muscle. In a specific embodiment according to the invention, prostate cancer is excluded from the cancers according to the invention.

C-cbl being an anti-apoptic regulator, the method of the present invention is preferably used for treating and/or preventing cancers that are associated with resistance to apoptosis. As used herein, the term “cancer associated with resistance to apoptosis” refers to a cancer that does not respond to conventional chemotherapy in which e.g. alkylating agents, antimetabolites, antimitotics, topoisomerase inhibitors, hormonal therapy drugs, aromatase inhibitors and/or signaling inhibitors are used. The c-cbl antagonist restores the capacity of the cells to enter apoptosis and thus restores the sensitivity of the cancer cells to such conventional chemotherapy agents. The man skilled in the art can easily determine whether a cancer is associated with resistance to apoptosis or not. Firstly, cancers associated with resistance to apoptosis do not respond any more to conventional chemotherapies. Secondly, proteins like Bcl-2 and iAPs are over-expressed in cancers associated with resistance to apoptosis and can thus be used as markers for determining whether a cancer is associated with resistance to apoptosis or not. In the frame of the present invention, it has further been found that over-expression of c-cbl is also a marker for resistance to apoptosis in cancer cells. Finally, resistance to apoptosis is linked with oxidative stress, which can readily be measured by the skilled in the art. Indeed, many methods for measuring oxidative stress in cancer cells are known in the art.

In a specific embodiment, the cancer associated with resistance to apoptosis is hormone-independent, i.e. it is a cancer that is defined clinically as hormone refractory and unresponsive. The cancer associated with resistance to apoptosis may for example correspond to an androgen-independent prostate, cancer, or to an estrogen-independent breast or ovary cancer.

As used herein, the term “c-cbl” refers to the Casitas B-lineage lymphoma proto-oncogene (SwissProt Accession No. P22681). In a preferred embodiment, c-cbl refers to the p120^{cbl} isoform. The sequence of an allele of the wild-type isoform p120^{cbl} is shown as SEQ ID NO: 1.

As used herein, the term “c-cbl antagonist” refers to a compound that inhibits or reduces c-cbl biological activity. In a preferred embodiment, the antagonist specifically inhibits the p120^{cbl} isoform. The biological activity of c-cbl depends on its concentration (i.e. its expression level) and on its specific activity. Therefore, the c-cbl antagonist may reduce or inhibit (i) c-cbl expression, (ii) c-cbl enzymatic activity (E3 ligase activity), and/or (iii) c-cbl poly-adaptor function, i.e., reduce or inhibit binding of c-cbl to at least one binding partner such as e.g. Grb2, EGF-R , CIN85, Sprouty and E2 ubiquitine, thereby reducing or inhibiting signal transmission within the signaling pathway. Preferably, the c-cbl antagonist in accordance with the invention reduces or inhibits c-cbl poly-adaptor function.

Methods for determining whether a compound is a c-cbl antagonist are well-known by the skilled in the art.

For example, the skilled in the art can assess whether a compound reduces or abolishes c-cbl expression by Western Blotting or by RT-PCR. The protocols provided in Example 1.5 may for example be used.

Alternatively, the E3 ligase activity of c-cbl in the presence of a compound may be compared to its E3 ligase activity in the absence of said compound. This may be done by measuring the capacity of c-cbl to ubiquinate RTKs (e.g. EGF-R), for example using the method described in Duan et al. (2003 J. Biol. Cell, 278:28950-28960), or by measuring the capacity of c-cbl to provoke endocytosis of RTKs (e.g. EGFR), for example using the method described in Kirisits et al. (2007 Int J Biochem Cell Biol. 39:2173-82). Typically, the capacity of c-cbl to ubiquinate EGF-R may be assessed by immunoprecipitating EGF-R and by performing a Western Blot using anti-ubiquitin antibodies. A compound reducing or abolishing the capacity of c-cbl to ubiquinate RTKs and/or to provoke endocytosis is defined as a c-cbl antagonist.

The biological activity of c-cbl may also be measured by assessing the capacity of c-cbl to bind to its natural binding partners such as e.g. Grb2, EGF-R, CIN85 or Sprouty (see e.g. Kirisits et al. 2007 Int J Biochem Cell Biol. 39:2173-82). The binding of c-cbl to Grb2, EGF-R, CIN85 or Sprouty may for example be assessed using an immunoprecipitation assay, a pull-down assay or the yeast two hybrid system (Y2H). A compound reducing or abolishing binding of c-cbl to Grb2, EGF-R, CIN85 and/or Sprouty is defined as a c-cbl antagonist.

The c-cbl antagonist may correspond to any type of molecule, such as e.g. a small molecule or a nucleic acid selected from the group consisting of an interfering RNA (iRNA), an antisense DNA and an aptamer.

The c-cbl antagonist preferably corresponds to an iRNA, in particular a siRNA. iRNAs specifically targeting c-cbl are well known in the art and include, e.g. the iRNAs described in Singh et al. (2007 Proc Natl Acad Sci USA; 104:5413-8), Mitra et al. (2004 J Biol Chem. 279:37431-5) and Zhou et al. (2004 Biochem Soc Trans. 32(Pt 5):817-21). iRNAs targeting c-cbl and/or kits for constructing such iRNAs may be purchased from e.g. Invitrogen or Qiagen. The iRNA may for example be an iRNA comprising or consisting of (i) the sequences of SEQ ID NO: 2 and SEQ ID NO: 3; (ii) sequences at least 80%, 85%, 90% or 95% identical thereto, or (iii) sequences comprising or consisting of fragments of at least 5, 10 or 15 nucleotides of SEQ ID NO: 2 and SEQ ID NO: 3. The c-cbl iRNA in accordance with the invention does not target genes homologous to c-cbl, such as e.g. cbl-b. In a preferred embodiment, the iRNA specifically targets the p120^{cbl} isoform.

By “effective amount” is meant an amount sufficient to achieve a concentration of peptide which is capable of preventing or treating the disease to be treated. Such concentrations can be routinely determined by those of skilled in the art. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, etc. It will also be appreciated by those of stalled in the art that the dosage may be dependent on the stability of the administered peptide.

By “individual in need thereof” is meant an individual suffering from or susceptible of suffering from the disease to be treated or prevented. The individual to be treated in the frame of the invention may correspond to any mammal. In a preferred embodiment, the individual is a human.

By “method of treating a cancer associated with resistance to apoptosis” is meant a method aiming at curing, improving the condition and/or extending the lifespan of an individual suffering from a cancer associated with resistance to apoptosis. By “method of preventing a cancer associated with resistance to apoptosis” is meant a method aiming at preventing the appearance of a resistance to apoptosis in an individual suffering from a cancer that is not yet associated with resistance to apoptosis.

The method of treating or preventing cancer according to the invention preferably corresponds to a combination chemotherapy. Indeed, the c-cbl antagonist according to the invention restores apoptosis and thus restores and/or enhances the efficacy of known agents currently used in chemotherapy. Thus the c-cbl antagonist may for example be administrated to an individual in combination with at least one of the following anti-cancer agents (simultaneously or sequentially):
an alkylating agent such as Cyclophosphamide, Chlorambucil and Melphalan;
an antimetabolite such as Methotrexate, Cytarabine, Fludarabine, 6-Mercaptopurine and 5- Fluorouracil;
an antimitotic such as Vincristine, Paclitaxel (Taxol), Vinorelbine, Docetal and Abraxane;
a topoisomerase inhibitor such as Doxorubicin, Irinotecan, Platinum derivatives, Cisplatin, Carboplatin, Oxaliplatin;
a hormonal therapy drug such as Tamoxifen;
an aromatase inhibitor such as Bicalutamide, Anastrozole, Examestane and Letrozole;
a signaling inhibitor such as Imatinib (Gleevec), Gefitinib and Erlotinib;
a monoclonal antibody such as Rituximab, Trastuzumab (Herceptin) and Gemtuzumab ozogamicin;
a biologic response modifier such as Interferon-alpha;
a differentiating agent such as Tretinoin and Arsenic trioxide; and/or
an agent that block blood vessel formation (antiangiogenic agents) such as Bevicizumab, Serafinib and Sunitinib.

In addition, the method of treating or preventing cancer according to the invention may be associated with a radiation therapy and/or surgery.

The invention also pertains to a c-cbl antagonist for use in activating and/or enhancing apoptosis, for example in cancer cells, and to a c-cbl antagonist for use in the treatment and/or prevention of a cancer associated with resistance to apoptosis.

Use of c-cbl as a Target for Screening for Cancer Drugs

A second aspect of the invention is directed to a method of screening for drugs for the treatment of a cancer, in particular a cancer associated with resistance to apoptosis, comprising the steps of:
providing a test compound; and
determining whether said test compound inhibits c-cbl;
   wherein the determination that said test compound inhibits c-cbl indicates that said test compound is a drug for the treatment or the prevention of cancer.

This method is preferably carried out in vitro or ex vivo.

More specifically, this method may comprise the steps of:
a) providing a test compound; and
b) determining c-cbl biological activity in the presence of said test compound;
c) determining c-cbl biological activity in the absence of said test compound; and
d) comparing the results of steps (a) and (b)
   wherein the determination that the biological activity measured at step (b) is lower than the biological activity measured at step (c) indicates that said test compound is a drug for the treatment or the prevention of cancer.

Preferably, said drugs for the treatment of a cancer is a drug for treating a cancer selected from the group consisting of prostate cancer (e.g. prostate adenocarcinoma), lung cancer (e.g. squamous cellular carcinoma), breast cancer (e.g. infiltrated ductal carcinoma), ovary cancer (e.g. serous papillary carcinoma), uterus cancer (squamous cellular carcinoma), brain cancer (e.g. astrocytoma), colon cancer (e.g. colon adenocarcinoma), colorectal cancer, rectal cancer (e.g. rectal adenocarcinoma), cancer of the striated muscle (rhabdomyosarcoma), thyroid cancer, testicular cancer, lymphoma and melanoma.

As presented hereabove, c-cbl biological activity may be measured by many methods well-known in the art, for example by measuring its expression level by Western Blotting or RT-PCR, by assessing its E3 ligase activity by measuring ubiquination or endocytosis of RTKs (e.g. EGF-R), or by assessing its binding to binding partners such as EGF-R, Grb2 and/or CIN85 using a yeast two hybrid system, a pull-down assay or immunoprecipitation.

The test compound may correspond to any type of compound. It may for example correspond to a small molecule or a nucleic acid selected from the group consisting of an interfering RNA, an aptamer and an antisense DNA. In a preferred embodiment, the test compound is a small molecule and a library of small molecules is screened with the method according to the invention.

The invention is also directed to the use of c-cbl as a target for screening for a c-cbl antagonist for the treatment of cancer, and to the use of c-cbl as a target for screening for a c-cbl antagonist decreasing resistance to apoptosis in cancer.

Use of c-cbl as a Diagnostic and/or Prognostic Marker in Cancer

The results presented herein show that human prostate tumor cells are characterized by elevated c-cbl expression levels when tested either by western blotting or by immunohistochemistry. Controls originating from the surrounding healthy tissue or from healthy prostate all appear to be very weakly labeled with c-cbl. Western blotting experiments showed that c-cbl expression levels were 2 to 6 times higher in prostate tumor than in the surrounding healthy tissues (FIG. 5).

In addition, the c-cbl expression level appears to reflect the degree of aggressiveness of the tumor, after correlating both of the western blot results and of the in situ labeling results with an anatomopathology analysis. Moreover, the expression level of c-cbl is different in prostate cancer and in benign prostatic hyperplasia (BPH).

Finally, as shown in Example 7, a higher expression level of c-cbl in tumoral tissues compared to healthy tissues was not only found in prostate cancer cells, but also in other cancers including lung cancer (e.g. squamous cellular carcinoma), ovary cancer (e.g. serous papillary carcinoma), uterus cancer (squamous cellular carcinoma), brain cancer (e.g. astrocytoma), colon cancer (e.g. colon adenocarcinoma), rectal cancer (e.g. rectal adenocarcinoma) and cancer of the striated muscle (rhabdomyosarcoma).

Therefore, a third aspect of the invention is directed to a method of diagnosing a cancer, in particular a cancer associated with resistance to apoptosis, comprising the steps of:
a) providing a biological sample from a patient susceptible of suffering from cancer;
b) determining c-cbl expression level in said biological sample; and
c) comparing the c-cbl expression level measured at step (b) with a value or a range of values measured in an unaffected biological sample;
   wherein the determination that the c-cbl expression level measured at step (b) is higher than the value or the range of values measured in the unaffected biological sample indicates that said patient suffers from cancer.

This method is preferably carried out in vitro or ex vivo.

The invention is further directed to the use of c-cbl for diagnosing cancer, in particular a cancer associated with resistance to apoptosis. More specifically, c-cbl is used as a marker for diagnosing cancer.

Preferably, said cancer is selected from the group consisting of prostate cancer (e.g. prostate adenocarcinoma), lung cancer (e.g. squamous cellular carcinoma), ovary cancer (e.g. serous papillary carcinoma), uterus cancer (squamous cellular carcinoma), brain cancer (e.g. astrocytoma), colon cancer (e.g. colon adenocarcinoma), colorectal cancer, rectal cancer (e.g. rectal adenocarcinoma) and cancer of the striated muscle (rhabdomyosarcoma). In a specific embodiment according to the invention, prostate cancer is excluded from the cancers according to the invention.

In a preferred embodiment, the determination that the c-cbl expression level measured at step (b) is at least 25 or 50% higher than the value or the range of values measured in the unaffected biological sample indicates that said patient suffers from cancer. Most preferably, the determination that the c-cbl expression level measured at step (b) is at least 2, 3, 4, 5, 6 or 7 times higher than the value or the range of values measured in the unaffected biological sample indicates that said patient suffers from cancer.

The c-cbl expression level may be determined using any method well-known in the art. For example, it may be determined by RT-PCR. Alternatively, it may be determined by immunohistochemistry. Such methods are described in details in the examples. The immunohistochemistry experiments may for example be performed using the Cbl (C-15) antibody, the Cbl (A-9) antibody or the Cbl (2111C3a) antibody that are commercialized by Santa Cruz Biotechnology (California, U.S.A). The antibody preferably corresponds to the Cbl (C-15) antibody.

Preferably, the unaffected biological sample corresponds to healthy tissue from the patient susceptible of suffering from cancer. Indeed, surrounding healthy tissue is the best control because the two samples can be taken and studied in parallel, during experiments carried out in parallel in identical conditions. One skilled in anatomopathology can easily differentiate abnormal tissue (i.e. potential cancerous tissue) from healthy surrounding tissue. In this embodiment, the diagnostic method in accordance with the invention comprises a further step (b2) of determining c-cbl expression level in healthy tissue from said patient, and step (c) comprises comparing the c-cbl expression level measured at step (b) and step (b2).

Alternatively, the unaffected biological sample may come from an unaffected individual. The value or a range of values of c-cbl expression measured in an unaffected biological sample may either have been determined prior to carrying out the diagnostic method in accordance with the invention, or be determined in the frame of the diagnostic method in accordance with the invention. When the value or ranges of values of c-cbl expression measured in an unaffected biological sample is determined prior to carrying out the diagnostic method in accordance with the invention, this value or range of values is preferably determined from data obtained from at least 2, 5, 10, 50 or 100 unaffected biological samples. When the value or ranges of values of c-cbl expression measured in an unaffected biological sample is determined in the frame of the diagnostic method in accordance with the invention, the diagnostic method in accordance with the invention comprises a further step of determining c-cbl expression level in an unaffected biological sample before performing step (c).

In a preferred embodiment, the biological sample from the patient susceptible of suffering from cancer preferably comprises epithelial cells and/or differentiated luminal cells. In addition to epithelial cells and/or differentiated luminal cells, the biological sample may comprise stromal cells as an internal control. The determination that c-cbl expression level is (i) higher in epithelial cells and/or differentiated luminal cells than in the healthy surrounding tissue; and (ii) higher in epithelial cells and/or differentiated luminal cells than in stromal cells indicates that said patient suffers from cancer. Indeed, it has been found that in prostate cancer, c-cbl is over-expressed in epithelial cells and/or differentiated luminal cells.

In another preferred embodiment, the diagnostic method in accordance with the invention is carried out to diagnose prostate cancer.

The above diagnostic method may be used e.g. for diagnosing cancer in an individual, for prognosing the outcome of the cancer, for designing a treatment regimen, for monitoring the progression of the cancer, and/or for monitoring the response of the individual to a drug (i.e. “drug monitoring”). More specifically, when the above diagnostic method is used to monitor the progression of a disorder and/or to monitor the response to a drug, it is repeated at least at two different points in time (e.g. before and after onset of a treatment).

It has been found that c-cbl expression levels are correlated with the grade of cancer cells. c-cbl may thus be used as a marker for determining the aggressiveness of a cancer, without the need of performing extensive anatomo-pathological studies.

The invention is thus directed to a method of diagnosing the aggressiveness of a cancer comprising the steps of:
a) providing a biological sample from a patient susceptible of suffering from cancer;
b) determining c-cbl expression level in said biological sample; and
c) comparing the c-cbl expression level measured at step (b) with values or ranges of values measured in biological samples from:
   i. individuals suffering from a non-aggressive cancer; and
   ii. individuals suffering from an aggressive cancer;

wherein:
the determination that the c-cbl expression level measured at step (b) is identical to the value or falls within the range of values measured in biological samples from individuals suffering from a non-aggressive cancer indicates that said cancer is not aggressive; and
the determination that the c-cbl expression level measured at step (b) is identical to the value or falls within the range of values measured in biological samples from individuals suffering from an aggressive cancer indicates that said cancer is aggressive.

This method may further comprise the step of comparing the c-cbl expression level measured at step (b) with values or ranges of values measured in biological samples from individuals suffering from benign prostatic hyperplasia.

The terms “aggressive cancer” and “non-aggressive cancer” are both well-known and clear to the skilled in the art. The aggressiveness of a cancer may for example be determined by determining the grade (G1-4) of the cancer cells. More specifically, cancer cells are “low grade” if they appear similar to normal cells, and “high grade” if they appear poorly differentiated. For example, a G1 cancer would be classified as a non-aggressive cancer, whereas a G4 cancer would be classified as an aggressive cancer. Additionally or alternatively, the aggressiveness of a cancer may be determined using the TNM classification. In this classification, T(a,is,(0),1-4) indicates the size or direct extent of the primary tumor, N(0-3) indicates the degree of spread to regional lymph nodes, and M(0/1) indicates the presence of metastasis. For example, a T1/N0/M0 cancer would be classified as a non-aggressive cancer, whereas a T4/N3/M1 cancer would be classified as an aggressive cancer.

High expression of c-cbl indicates that the cancer cells have become resistant to apoptosis. Therefore, patient having such cancer cells needs to be treated by an aggressive therapy. C-cbl can thus be used as a marker for selecting the treatment regimen of a patient.

The invention is thus directed to a method for selecting a patient suffering of a cancer suitable to be treated by an aggressive chemotherapy comprising the step of determining c-cbl expression level in a biological sample from said patient, and selecting the patient if it has a high expression level of c-cbl.

By “patient having a high expression level of c-cbl” is meant a patient having a c-cbl expression level that is at least 25 or 50% higher, and preferably at least 2, 3, 4, 5, 6 or 7 times higher, than the value or the range of values of c-cbl expression level in an unaffected individual and/or in a sample of healthy tissue from the patient.

By “aggressive chemotherapy” is meant a chemotherapy adapted for treating aggressive cancers. Specifically, such aggressive chemotherapies may induce side effects and do therefore not constitute the preferred treatment regimen in the case of a non-aggressive cancer. An aggressive chemotherapy typically corresponds to a combination chemotherapy carried out with high doses of drugs. The combination chemotherapy may for example comprise the administration of high doses of at least one compound selected from the group consisting of an alkylating agent, an antimetabolite, an antimitotic, a topoisomerase inhibitor, a hormonal therapy drug, a signaling inhibitor, an aromatase inhibitor, a differentiating agent, a monoclonal antibody, a biologic response modifier and an antiangiogenic agent. The aggressive chemotherapy may further be combined with a radiation therapy and/or surgery.

In a preferred embodiment, the aggressive chemotherapy comprises the administration of a c-cbl antagonist.

The invention is also directed to the use of c-cbl as a marker for selecting a patient to be treated with a c-cbl antagonist, and to a method for selecting a patient suffering of a cancer suitable to be treated by a c-cbl antagonist comprising the step of determining c-cbl expression level in a biological sample from said patient, and selecting the patient having a high expression level of c-cbl.

The invention is further directed to a method of treating or preventing a cancer associated with resistance to apoptosis comprising the steps of:
a) determining c-cbl expression level in a biological sample from said patient;
b) selecting the patient having a high expression level of c-cbl; and
c) administering an effective amount of a c-cbl antagonist to said patient having a high expression level of c-cbl.

All references cited herein, including journal articles or abstracts, published patent applications, issued patents or any other references, are entirely incorporated by reference herein, including all data, tables, figures and text presented in the cited references.

Although having distinct meanings, the terms “comprising”, “having”, “containing' and “consisting of” have been used interchangeably throughout this specification and may be replaced with one another.

The invention will be further evaluated in view of the following examples and figures.

### BRIEF DESCRIPTION OF THE FIGURES

FIG. 1 represents the results of c-cbl co-amplification RT-PCR (upper line) and c-Cbl Western Blottings (lower line) of Ventral Prostate from adult Rats (90 days post natal) exposed to flutamide for different durations (CT: control; 24, 48, 72 and 96 hours).

FIG. 2 shows the results of c-Cbl Western Blottings in mouse prostate at days 16, 17, 18 and 20 after birth. The gene used for normalization of expression level is CK18.

FIG. 3 A. Expression of Bim EL in c-Cbl KO and wild type (WT) MEFs. The cells were either untreated (CTRL), or treated with 0.1 mM Etoposide (Etop), or with 1 μM Hydrogen Peroxyde (H₂O₂) for 24 hours. B. Expression of c-IAP2 (left) and XIAP (right) in MEF after the same treatment. Expression was studied by Western blotting. The lower part of the columns represents the average value, and the upper part of the columns represents the standard deviation.

FIG. 4. A. Expression of activated Caspase-3 in c-Cbl KO and WT MEFs treated with 0.1 mM H₂O₂ or 1 μM Etoposide for 24 hours. B. Nuclear fragmentation revealed by DAPI experiments of the c-Cbl KO and WT MEFs after 16H or 24H treatment with various concentrations of Etoposide (1 or 10 μM) or H₂O₂ (0.1 or 0.5 mM).

FIG. 5 represents c-Cbl expression in human prostate tumor compared to the expression in normal tissue from the same patient. Samples from six different patients (P1 to P6) were analyzed. C-cbl expression was studied by Western blotting.

FIG. 6 shows the high c-Cbl expression in various tumours compared to normal surrounding tissue. In A to H, the left panel shows a normal tissue and the right panel shows a tumour tissue corresponding of the same origin. All tissues were stained with an anti-c-Cbl antibody through immunohistochemistry experiments. Tissue Microarrays (TMA) were investigated and spots of at least six different patients were compared, showing equivalent results. A. prostate versus prostate adenocarcinoma. B. breast versus infiltrated ductal carcinoma. C. ovary versus serous papillary carcinoma. D. uterus versus squamous cellular carcinoma. E. brain versus astrocytoma. F. lung versus squamous cellular carcinoma. G. colon versus colon adenocarcinoma. H. rectum versus rectum adenocarcinoma. The magnification bar represents 50 μm.

FIG. 7 shows that c-Cbl expression level is decreased upon treatment with H202 or etoposide in LNCaP cells and that silencing of c-Cbl reduces the endonuclease oxidative stress. A. c-Cbl expression upon a 24 hours treatment with 25 or 50 nM of hydrogen peroxide respectively, or with 10 or 30 μM of etoposide respectively. Expression of the anti-apoptotic Bcl-2 and c-IAP2 proteins and of the pro-apoptotic Bcl-2 family protein Bax is also shown. B. c-Cbl expression after 48 hours of c-Cbl silencing. Expression of APE1, which is indicative of oxidative stress, is shown compared with medium control (Ctrl) and with si-control (si-Ctrl).

FIGS. 8 and 9 show that the mitochondrial apoptosis pathway of mouse ventral prostate (VP) is up-regulated by c-Cbl. The western-blotting analysis was done with the ventral prostate tissue of c-Cbl KO and WT mice, 30 days after birth (young adults). Actin is the protein of reference and each western blotting panel is representative of three independent experiments. FIG. 8A: proapoptotic Bim EL protein expression in c-Cbl KO and wild type (WT) mouse VP. Bim is significantly higher in KO mice (p=0.048). FIG. 8B: proapoptotic Bak protein expression in c-Cbl KO is higher than in WT mouse VP. FIG. 8C: Smac/Diablo expression in c-Cbl KO is significantly superior to WT mouse VP (p=0.0183). FIG. 8D, E and F: c-IAP1, c-IAP2 and XIAP expressions in c-Cbl KO and WT mouse VP. C-IAP1 is significantly lower in KO VP (p=0.0201) as well as XIAP (p=0.035). On FIGS. 8A to 8F, the vertical axis represents the expression level of the studied gene on the expression level of Actin. FIG. 9A: processed caspase 9 expression in c-Cbl KO and WT mouse VP. Activated caspase 9 was higher in KO VP. FIG. 9B: number of apoptotic cells from TUNEL experiments made on c-Cbl KO and WT mouse VP tissue sections. The number of apoptotic cells from flutamide treated animals is also indicated. KO VP supports significantly more apoptotic cells than WT (p=0.0014).

FIG. 10A. Expression of activated Caspase-3 in c-Cbl KO and WT MEFs treated with H₂O₂ or Etoposide. B. Number of apoptotic cells from TUNEL experiments made on c-Cbl KO and WT MEFs treated with H₂O₂ or Etoposide. MEF cells that are knocked-out for c-cbl are about twice more sensitive to H₂O₂ than MEF cells that are wild-type for c-cbl.

### BRIEF DESCRIPTION OF THE SEQUENCES

SEQ ID No. 1 corresponds to the amino acid sequence of human c-cbl (p120^{cbl}).

SEQ ID Nos. 2 and 3 correspond to iRNAs inhibiting c-cbl.

SEQ ID Nos. 4 and 5 correspond to the primers used for verifying that c-cbl is not expressed in c-Cbl KO mice.

SEQ ID Nos. 6 and 7 correspond to the primers used for amplifying c-cbl.

### EXAMPLES

### Example 1

### Materials and Methods

1.1. Mice

The experiments were carried out either in vivo on rats (on knock-outs for c-cbl and on wild-type mice having the same genetic background, sv129) or in vitro on MEFs originating from mice knockouts for c-cbl or from wild-type mice which we produced on embryonic day 13. The c-cbl-/-(KO) animals were produced starting from mice having an sv129 genetic background (Naramura et al. 1998 Proc Natl Acad Sci USA. 1998 95:15547-52). Sprague Dawley rats were also used (IFFA Credo, l'Arbresle, France).

1.2. Cell Lines and Tumors

Biological samples from human prostate cancer tumors were supplied by Dr. Myriam Decaussin-Petrucci (Lab. Anat. Cytol. Pathol., CHU Lyon-Sud, Lyon).

LNCaP is a human cell line, derived from a prostatic hormone-dependent metastatic tumor. The cells were maintained in RPMI 1640 medium (Invitrogen), supplemented with 7.5% FCS, 20 μg/ml streptomycin, 20 U/ml nystatin. LNCaP were used between the passages 50-60. For western-blotting LNCaP cells were seeded in 10cm disk (22.10⁵ cells) and 6 cm disk (8.10⁵ cells) respectively, with 5 disks per condition. Cells were allowed to attach for 24H and treated with various concentrations of R1881 at day 0. All the cells, including the controls, were cultured in the presence of the same ethanol concentration. For H₂O₂ or etoposide treatment, LNCaP cells were cultured for 24 hours in the presence of known concentrations of H₂O₂ or etoposide. The RNA silencing was classically done in Optimen medium (Gibco) using lipofectamine 2000 following the manufacturer's instructions (Invitrogen). The c-Cbl RNAi was generated by Eurogentec. Its sequence is: GGGAAGGCUUCUAUUUGUU (SEQ ID NO: 2). The siRNA controls were purchased from Invitrogen : siRNA-A (sc-37007) and siRNA-B (sc-44230).

The RAT1-MEN2A cell line has been used. This cell line is an immortalized but untransformed rat fibroblast line in which chimeric Ret receptor tyrosine kinases and also the coreceptor for Ret, GFR alpha, are overexpressed. The chimeric Ret has, in the C-terminal intracytoplasmic region, an Fv sequence capable of binding transiently to the chemical product AP. Thus, in the presence of AP, there is dimerization of Ret-Fv and oncogenic-type activation. On the other hand, in the presence of the physiological ligand for Ret, GDNF, there is formation of a transient tetramer (Ret-Fv)₂+(GFRalpha)₂, which leads to physiological-like activation.

1.3. Chemicals and Antibodies

Flutamide, obtained from Aldrich Chemical Co., was dissolved in an aqueous solution of methylcellulose 400 (Fluka) at 0.5% (w/v). Testosterone agonist methyltrienolone (R188) was purchased from NEN Life Science Products. Protease inhibitor cocktail was obtained from Roche Molecular Biochemicals (Mannheim, Germany). Hydrogen peroxide, Etoposide, 4′, 6′-diamidino-2 phenylindole (DAPI), actin polyclonal antibody, Tween 20 and Biomax MR film were obtained from Sigma. Schleicher & Schuell. Polyvinyl difluoride (PVDF) membranes were purchased from Merck Eurolab (Strasbourg, France). Horseradish peroxidase-labelled anti-rabbit IgG and the chemiluminescent kits were obtained from CovalAb. TRIzol and dNTPs were obtained from Life Technologies. Taq polymerase was purchased from Promega Life Science. Primers were synthesized either by ProligoFrance SAS (Paris, France) or by MWG GmbH (Ebersberg). M-MLV and [³³P]dATP (1000-3000 Ci/mmol) were purchased from Amersham (Orsay, France). Mayer's haematoxylin and the aqueous mounting medium (Faramount) were obtained from Dako (Trappes, France).

The antibodies used in the frame of the examples were the following commercially available antibodies: an anti-cbl antibody directed against the 15 C-terminal amino acids of c-cbl were obtained from Santa Cruz Biotechnology (California, U.S.A, Catalogue No. sc 170), an anti-Cbl-b antibody (Santa Cruz, Catalogue No. C 20), a mouse polyclonal raised against cytokeratin 18 (CK18), a rabbit polyclonal raised against Androgen Receptor (AR, sc-815), an anti-Bim antibody (Santa Cruz, Catalogue No. H 191), an anti-Smac/DIABLO antibody (Santa Cruz, Catalogue No. V 17), an anti-Caspase 9 antibody (Santa Cruz, Catalogue No. H 83), an anti-activated Caspase 3 or 6 antibody (Ozyme, catalogue Nos. Covalab and N0 9762 respectively), an anti-c IAP1 or 2 antibody (Santa Cruz, Catalogue No. H 85), an anti-XIAP antibody (Abcam, Catalogue No. ab21278), an anti-Bcl 2 antibody (Santa Cruz, Catalogue No. sc 492), and an anti-Akt and phAkt antibody (Santa-Cruz). The Apurinic/apyrimidinic endonuclease (APE1/REF1) human fusion protein rabbit polyclonal antibody was obtained from Abcam (Catalogue No. ab82).

1.4. iRNAs

The c-cbl IRNA was produced by Eurogentec and had the following sequences: 5′ GGGAAGGCUUCUAUUUGUU 3′ (SEQ ID NO: 2) and 5′ CUGUCCAUCUAGAGACAAA 3′ (SEQ ID NO: 3). It is effective on human, rat and mouse c-cbl. It is ineffective on cbl-b.

1.5. Western Blotting and Immunohistochemistry Experiments

The western blotting (WB) experiments, immunohistochemistry (IHC) experiments and RT PCR experiments, with coamplification of the S 20 ribosomal gene, were carried out according to conventional methods already described in e.g. Omezzine et al. (2003, Biol. Reprod., 69: 752-760), Bozec et al. (2004, J. Endocr., 183: 79-90) and El Chami et al. (2005, J. Cell. Biol., 651-661).

The primers for the RT-PCRs came from ProligoFrance or from MWG-Biotechnology. Primers used for c-cbl amplification had the following sequences: ATGGACAAGGTTGGTGCGGTTGTGGT (SEQ ID NO: 6) and GAAGAGGCTGATAGTCTGCTTAGT (SEQ ID NO: 7), thereby producing a 213 bp amplification product.

Western blotting experiments and immunohistochemistry experiments (with anti-c-cbl, anti-IAP2 and/or anti-XIAP antibodies) were carried out on human samples. The dilutions of the c-cbl antibody (Santa Cruz Biotechnology, California, U.S.A, Catalogue No. sc 170) were 1/200 for the IHC experiments and 1/3000 for the WB experiments.

The immunohistochemistry experiments corresponded to automated immunohistochemistry experiments. The IHC procedure was performed with a Ventana Benchmark XT autostainer using the manufacturer's procedure. Briefly, after paraffin removal, the slides were submitted to antigen retrieval with Cell Conditioner for 30 min at 95° C. Slides were then incubated for 32 min at 37° C. with specific primary antibodies. Ventana kits including the biotin/avidin/phosphatase system with Fast Red as chromogen was used and the slides were then counterstained with hematoxylin before mounting. Negative controls were obtained by omitting the specific primary antibodies

For analysis of immunohistochemical staining, the intensity was rated as none (−), weak (+), moderate (++), or intense (+++) for each slide. Specimens were considered immunopositive when 1% or more of the tissue had clear evidence of immunostaining. The immunostaining was evaluated by two independent observers in the laboratory, blinded as to the treatment status.

The images were acquired using a microscope (Axioskop; Carl Zeiss Microlmaging, Inc.) with plan-Neofluar objective lenses (Carl Zeiss Microlmaging, Inc.) at 40×/NA 0.75. Observation was performed with a 3,200-K halogen light plus a daylight blue filter using digital imaging medium. DAB was used as chromogen. The camera (Coolpix 990; Nikon) used the Nikon acquisition software. All manipulations were performed at room temperature. Image processing was performed with Adobe Photoshop, and only the whole images were processed with brightness, contrast, and color balance adjustments.

1.6. TUNEL and IHC Experiments

The TUNEL and also IHC experiments were carried out on sections of 5 micron originating from rat, mouse or human prostate. The samples paraffin-embedded after treatment in Bouin's solution, in formol, and subsequently dehydrated with graduated ethanol baths. The sections were subsequently deparaffinized (xylene), rehydrated in successive water/ethanol baths and then treated at 93-98° C. for 20 minutes in the presence of citric acid (epitope unmasking).

1.7. In Vivo Experiments

Rat or mice were treated with the testosterone antagonist flutamide (Aldrich Chemicals) dissolved in an aqueous solution of methylcellulose 400 (Fluka). Flutamide was administered orally to rats or mice (aged between 60 and 90 days) for 4 consecutive days at the dose of 10 mg/kg/day. The prostate lobe samples were taken the day after the flutamide treatment had been stopped.

Testosterone (testosterone heptylate 10 mg/kg, Theramex) was administered to rats castrated one day beforehand by subcutaneous injection at the dose of 1.6 mg/kg per day for 4 consecutive days.

1.8. In Vitro Experiments

The testosterone agonist R1881 (Life Science Products) was used in LNCaP cell cultures at various concentrations (from 10⁻¹² M to 10⁻⁸ M).

The MEFs KO for c-cbl and the MEFs WT were cultured in DMEM, 10% FCS. The apoptosis inducers were used at a final concentration of 0.5 mM in case of H₂O₂, and at 10 μM in case of etoposide. The cells were tested for apoptosis 24 hours after treatment. The human prostate cancer lines were transfected with 125 nM of c-cbl IRNA for 6 hours, and were tested for quenching of c-cbl expression 24 or 48 hours after transfection. The treatment was the same with RAT1-MEN2A cells.

1.9. Statistical Analyses

Data were expressed as the mean S.D. Three to seven animals from different litters were used. For statistical analysis of data generated in both in vivo and in vitro models, one-way ANOVA was performed to determine whether there were differences between all groups (P<0.05), and then the Bonferroni post-test was performed to determine the significance of the differences between the pair of groups. P<0.05 was considered significant. The statistical tests were performed on StatView software version 5.0 (SAS Institute Inc.).

### Example 2

### c-Cbl Expression is Androgen-Dependent in Rat Ventral Prostate

It is known in the art that the prostate organ in rodents is divided in three lobes, the Ventral Prostate (VP) being androgen-dependent, whereas the Cranial Prostate (CP) and Dorsal Prostate (DP) are not (Banerjee et al. 1995 Endocrinology. 136:4368-76).

Flutamide is known to induce apoptosis whose intensity depends on the dose of flutamide used (Kassim et al. 1997 J Anat. 190(Pt 4):577-88). The anti-androgen flutamide competes with Dihydotestosterone (DHT) (which is generated from testosterone by the 5alpha-reductase enzyme in prostate) at the Androgen nuclear Receptor (AR) level. The adult rats were first treated by the flutamide at a dose of 10mg/kg/day for various lengths of treatment and the different prostate lobes were analyzed for the expression of c-Cbl. Dose/effect measurements were also carried out. Secondly, castrated rats, whose main source of endogen testosterone is destroyed, were sacrificed a day after surgery for c-Cbl expression testing. Other animals were analyzed after a substitutive testosterone treatment given from day two to day five after surgery. Finally, in situ experiments were done allowing to finely localize c-Cbl in prostate and importantly to observe the impact of Flutamide on the c-Cbl expressing cells.

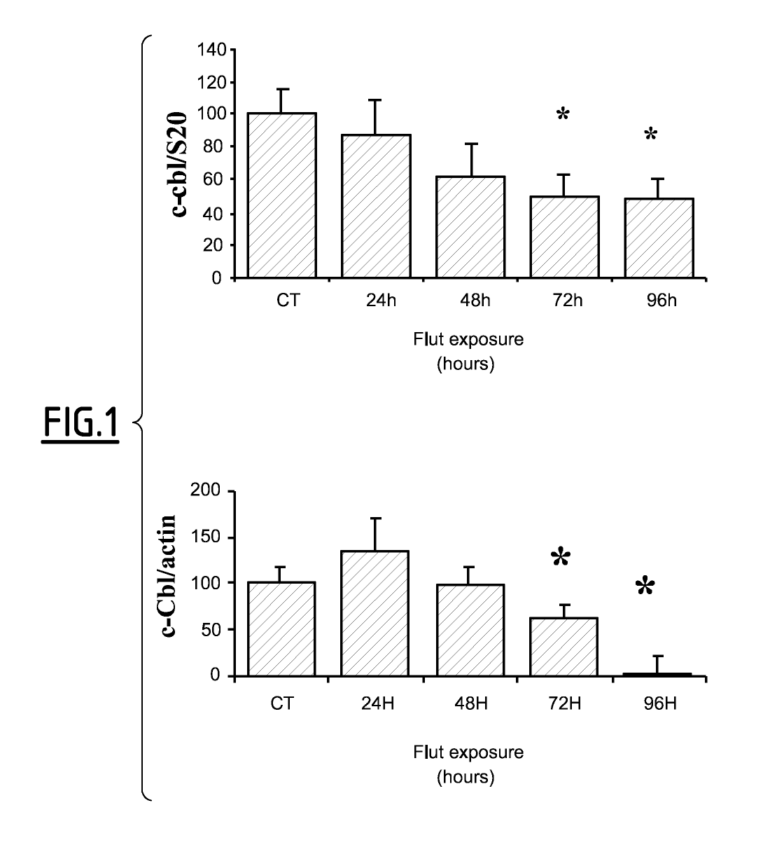

c-Cbl protein expression in ventral prostate was significantly lower (about one half) after three days of flutamide force-feeding than in the control (FIG. 1). The mRNA expression was also affected after two days of treatment, pointing to the fact that c-Cbl alteration is at the transcriptional level. However, the protein expression appeared to be more affected that the mRNA (96 H of treatment), which could be due for a part to a post-transcriptional effect or/and to a weak c-Cbl expression in prostate stroma cells. These results clearly showed that the inhibition of androgen activity results in a decreased expression of c-Cbl in ventral prostate.

The caudal and dorsal prostate lobes were also tested, but no difference of c-Cbl expression was seen whatever the length of flutamide treatment. These results reinforce the hypothesis that the expression of c-cbl goes through activated androgen receptors only when the tissue where it is expressed is androgen dependent itself. Indeed, it is known that the caudal and dorsal prostate lobes in rodent are not affected by androgens (Banerjee et al. 1995 Endocrinology. 136:4368-76). It is possible to consider that caudal and dorsal prostate are internal negative controls for the observed androgen dependency of c-Cbl in ventral prostate.

Dose/effect experiments were carried out in order to confirm that the c-Cbl decreased expression depends on the dose of flutamide, and in order to estimate at what dose of flutamide it could be possible to see a significant alteration of c-Cbl expression. The c-cbl decrease expression was indeed observed from the dose 2 mg/kg/day and diminished to one third time of the control at 10 mg/kg/day. It is important to note that such doses had a very weak impact on the number of living cells.

Castration performed on rats confirmed the androgen dependency of c-Cbl in ventral prostate. Specifically, as soon as a day after surgery one could observe a drop of c-Cbl expression. In the case of a substitutive testosterone treatment given during four days beginning the day after surgery, a quasi-normal c-Cbl expression level was observed. This result strongly suggests that c-Cbl is re-expressed to a physiological level, since four days of androgen treatment after surgery is quite short to repopulate the affected tissue. Once again, it points to the c-Cbl androgen-dependency in ventral prostate.

In situ experiments showed that c-Cbl is essentially expressed in the epithelial border of ventral prostate in rats. The differentiated luminal cells were stained, but not the basal luminal cells. The stroma cells were not stained neither. Coherent with the data reported above, the flutamide treatment led to an almost complete extinction of the c-Cbl staining at 96 H of 10mg/kg/day of treatment compared to the untreated control, stressing again the androgen dependency of c-Cbl expression. Very importantly, this dose did not affect at all the integrity of the epithelium border, allowing the comparison at both the protein and the mRNA level of prostate extractions from animals treated with such doses. This experiment also ensured that four days of treatment do not affect those luminal c-Cbl expressing cells. c-Cbl staining in the same in situ experiment was moderate in the luminal cells cranial prostate compare to ventral prostate, and not seen at all in the dorsal prostate. It is noticeable that the Androgen Receptor (AR) was strongly expressed in the epithelial cells of the ventral prostate, co-localizing with c-Cbl. As for c-Cbl, AR staining was switched off after flutamide treatment, particularly after a 96 hours treatment. This result underlines the tight relationship of activated AR and the c-Cbl expression in luminal prostate cells.

### Example 3

### Appearance of c-Cbl Androgen-Dependency During Maturation of Mouse Prostate

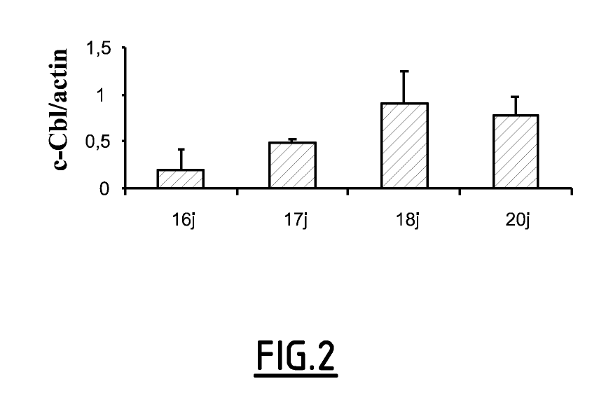

In order to explore the androgen dependency of c-Cbl in ventral prostate in mice, c-Cbl expression was analyzed during the mouse prostate development. It is known that the mouse prostate maturation indeed depends on the first wave of testosterone that appears around day 15 post-natal, as for any androgen dependent tissue (Chung 1995 Cancer Surv. 23:33-42). It could thus be possible then to detect the variation of the c-Cbl level expression from day 16 to day 20. The experiment was thus done with the epithelium specific marker Cytokeratin 18 (K18) (Schalken and van Leenders 2003 Urology. 62(5 Suppl 1):11-20), allowing comparing the c-Cbl expression levels from a day to another (FIG. 2). The c-Cbl expression increased more that four times from day 16 to days 18 or 20, which corresponds to the first wave of testosterone in mice.

This clearly proves the androgen dependency of the c-Cbl expression in mice.

### Example 4

### Flutamide-Induced Apoptosis in Rat Ventral Prostate is Associated with c-Cbl Down-Regulation

As the level of Androgen Receptor activation is correlated with the survival of epithelial cells in prostate and as c-Cbl expression is dependent on this effect, the relationship of c-Cbl with prostate cell apoptosis was next studied.

Investigations were first realized on rat ventral prostate to ensure that the c-Cbl down-regulation upon flutamide treatment was accompanied by an imbalance of the mitochondrial apoptotic pathway, which promotes its up-regulation. Two apoptotic markers which are altered in mouse testicular germ cells from c-Cbl KO mice were studied: the pro-apoptotic BH3-Only protein Bim EL and the Inhibitor of Apoptosis c-IAP2 (Uren et al. 2007 J Cell Biol 177:277-87; Strasser et al. 2005 Nat Rev Immunol 5:189-200; Schimmer 2004 Cancer Res 64:7183-90). Flutamide treatment with 10 mg/kg/day resulted in an increase of Bim EL expression (studied by western blotting). The increase was detected at 72 hours and was significant at 96 hours of treatment. In situ examination showed a complete absence of staining for the untreated control and a clear appearance of Bim EL staining of the ventral prostate luminal cells from 24 hours of treatment, co-localizing with c-Cbl and AR. The weak discrepancy between in situ experiments and western blotting relative to the duration of treatment needed increase Bim EL expression is probably linked to the difference of sensitivity of the two approaches. This is coherent with the increase of the proapoptotic Bim marker expression when c-Cbl expression decreases as already reported by El Chami et al. (2005; J Cell Biol. 171:651-61). Conversely, c-IAP2 expression was half time lower at 48 hours of flutamide treatment and stayed level after longer treatment. These data are in accordance with the initiation of apoptosis already described in literature for rat ventral prostate by Omezzine et al. (2003, Biol. Reprod., 69: 752-760).

These results show the tight association between the decrease of c-Cbl expression and the apoptotic initiation in prostate.

### Example 5

### c-Cbl Down-Regulates Apoptosis in Mouse Embryonic Fibroblasts Through the Mitochondrial Pathway

As c-Cbl appears to be a down regulator of luminal cell apoptosis in ventral prostate, a comparative study in Mouse Embryonic Fibroblasts (MEF) from KO and WT mice was realized.

As MEF cells did not express the Androgen Receptor, the Etoposide compound and the Hydrogen Peroxyde (H₂O₂) were used as apoptotic inducers. Each of them are known to involve different signaling routes, both leading to the mitochondrial pathway of apoptosis. Hydrogen peroxide activates C-Jun Kinase, whereas Etoposide blocks topoisomerase II causing dsDNA breaks and DNA-PK/p53 activation (DeYulia et al. 2005 Proc Natl Acad Sci USA 102:5044-9; Kamata and Hirata 1999 Cell Signal 11:1-14; Karpinich et al. 2002 J Biol Chem 277:16547-52).

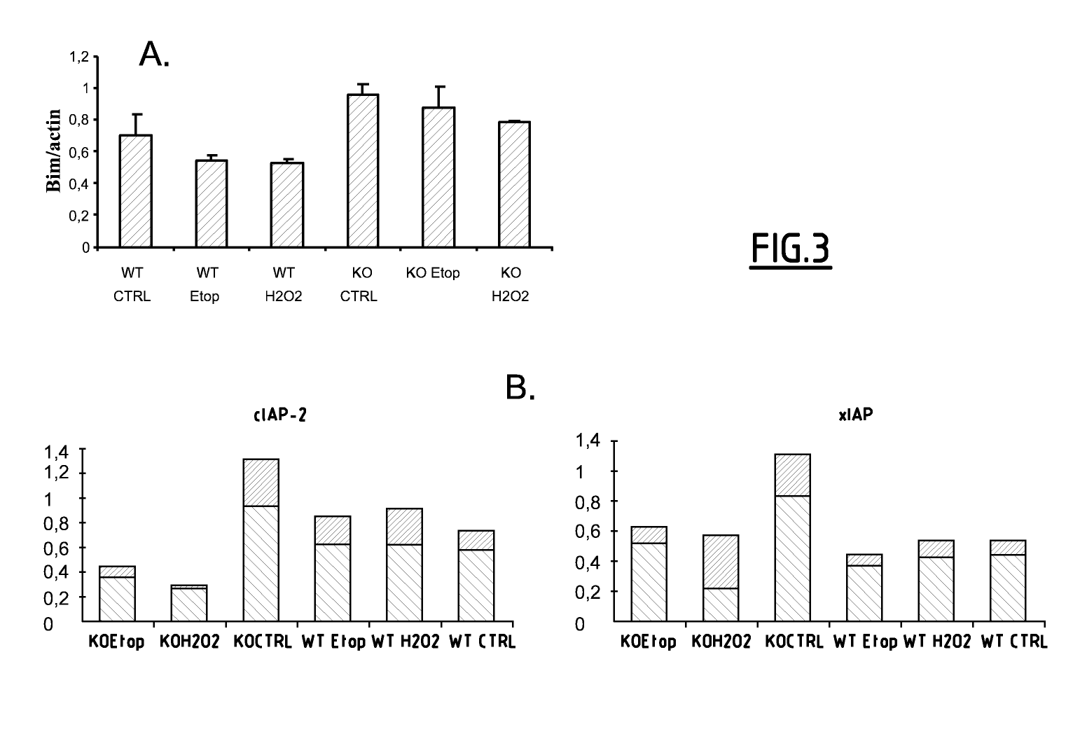

It was found that Bim EL was up-regulated in c-Cbl KO MEFs compared to WT MEFs (FIG. 3A). Interestingly, as in prostate luminal cells, apoptotic activation by flutamide did neither increase Bim EL expression in KO nor in WT cells. Thus c-Cbl does not particularly interfere with Bim when apoptosis is running. The c-IAP2 and XIAP Inhibitors of Apoptosis showed a similar level of expression level in KO and in WT untreated controls (FIG. 3B). The apoptosis activation by H₂O₂ and Etoposide initiates a significant decrease of these IAPs for around one/third of the control level.

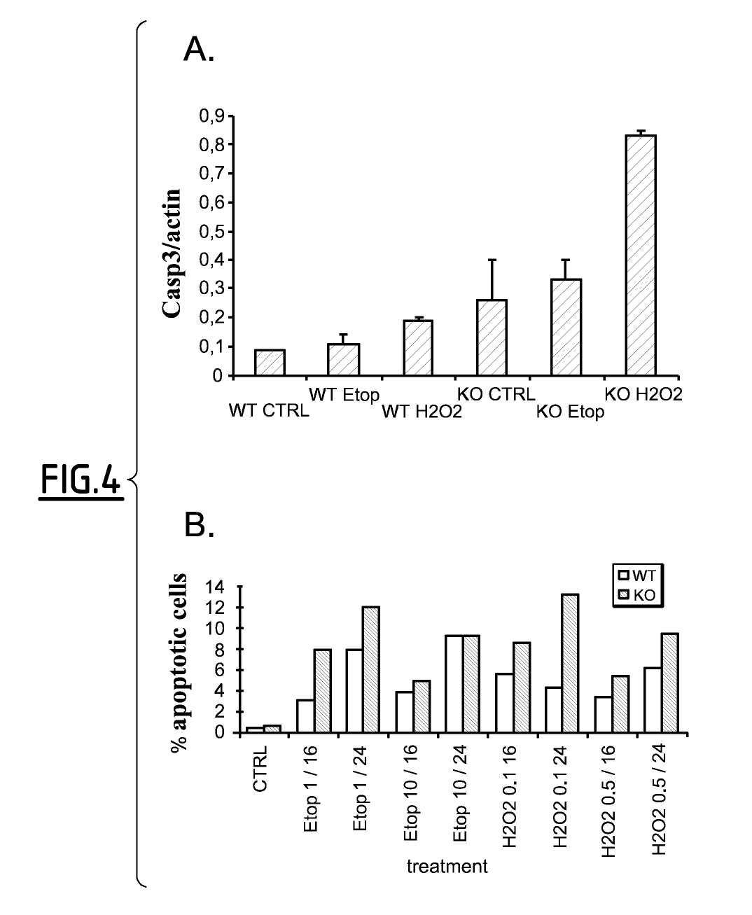

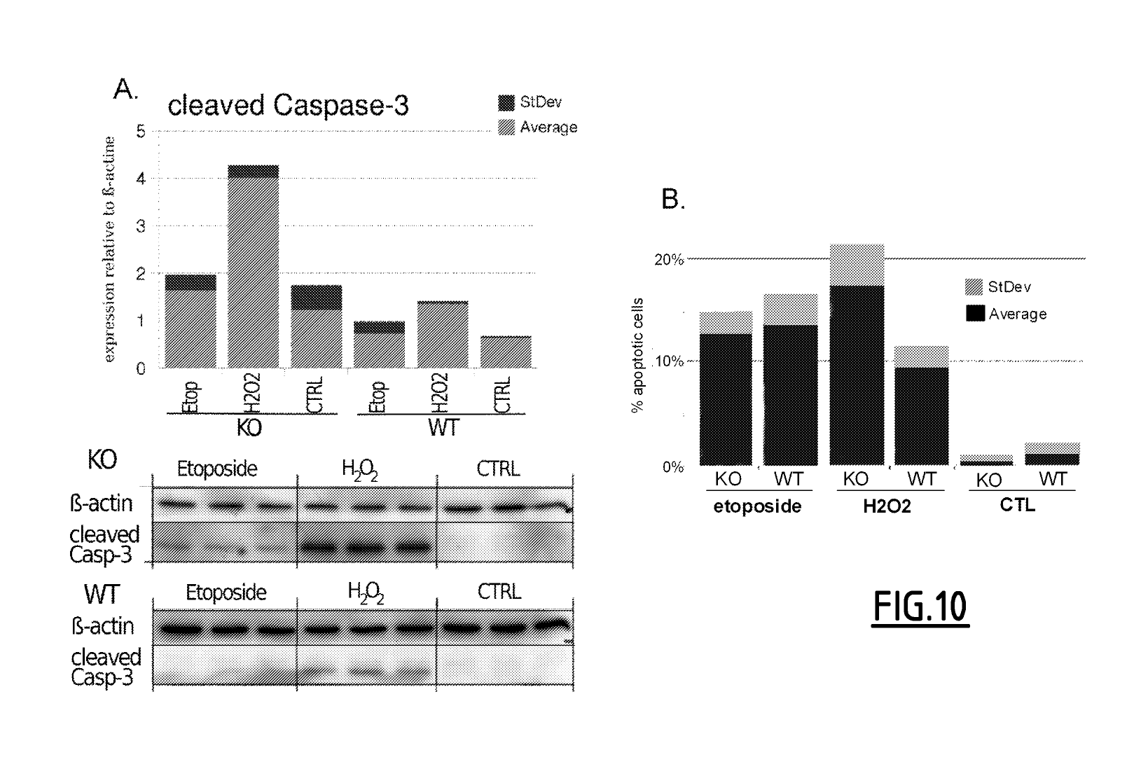

The apoptotic status of these cells was then tested. Cleaved (activated) Caspase 3 had a spontaneous significant expression two times higher in c-Cbl KO cells than in WT cells (FIGS. 4A and 10A). The Etoposide activation led to a slight but not significant increase of cleaved caspase 3 in either case (KO or WT), but led to a drastic, significant expression of the caspase effector in c-Cbl KO cells stimulated by hydrogen peroxide (three times and half the control). In comparison, when stimulated by H₂O₂, activated caspase 3 expression increased two times in WT, but still at a quite lower level than in KO. Etoposide had a slight effect over caspase 3 activation of MEFs, whereas the hydrogen peroxide activation involved a tight c-Cbl relationship in these cells. The percentage of apoptotic cells was indeed on average largely higher with H₂O₂ in KO MEF cultures than in WT cultures (43% more apoptotic cells), irrespective of the dilution of hydrogen peroxide used (0.1 to 0.5 nM) and of the time of activation in culture (16 or 24 H) (FIGS. 4B and 10B). Etoposide treatment was responsible in average of a weaker difference between KO versus WT apoptotic cells (29% more KO apoptotic cells). WT MEF cultures were slightly less sensitive to H₂O₂ than Etoposide treatment (4.9% of WT apoptotic cells versus 5.8% of KO apoptotic cells), whereas c-Cbl KO cultures were subjected to a higher difference between Etoposide and H₂O₂ treatment: 4% of apoptotic Etoposide-treated KO cells versus 8.75% of apoptotic H₂O₂-treated KO cells. The spontaneous apoptosis for both untreated cell types was quite low and could not be quantified.

In summary, in terms of number of apoptotic cells, the silencing of c-Cbl gave more sensitivity to cells upon hydrogen peroxide treatment than upon etoposide treatment. This aspect fully reflects the over-activation of Caspase 3 reported above. It is noteworthy that these cells must be stimulated for apoptosis to reveal the apoptotic imbalance, which corresponds to the very few number of cells spontaneously entering the apoptotic process. Moreover, all these results confirm the role of c-Cbl as a down-regulator of apoptosis in response to oxidative stress.

### Example 6

### c-Cbl Up-Regulation is Strongly Associated to Human Prostate Tumors

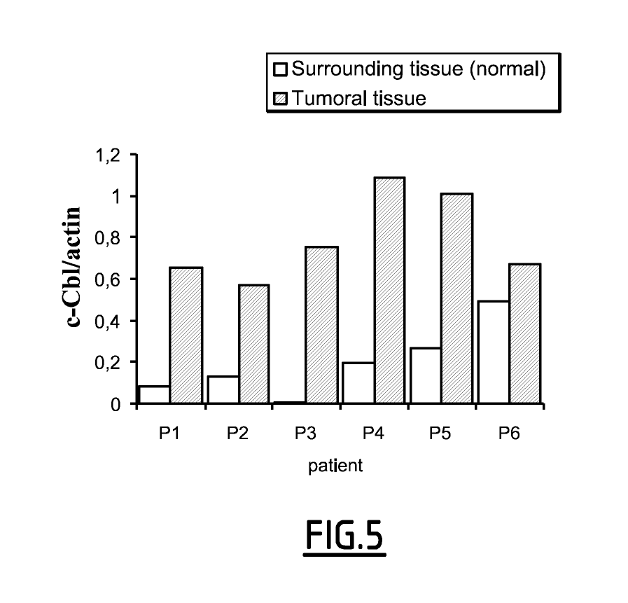

Prostate cancer sustains a well-known resistance to apoptosis (Denmeade et al. 1996 Prostate 28:251-65). Since it has been found in the frame of the present examples that c-cbl has a role in the down-regulation of apoptosis, the expression status of c-Cbl in prostate cancer was next explored by C-cbl expression was studied by Western blotting. Patients of T3 grade were subjected to surgery and samples were taken. Normal tissues were compared to cancer tissues of the same patient. These patients were not treated, neither by chemotherapy nor by radiotherapy. c-Cbl protein was drastically increased in tumor cells reaching at least four times the physiological control for almost all the analysis (FIG. 5).

Further immunohistochemistry experiments were then carried out. Several cases of human prostate adenocarcinoma were accumulated and listed according to their grade. The magnitude of the expression of c-cbl was measured for these tumors. Sections from benign prostatic hyperplasia (BPH) were also accumulated. The clinical and anatomical pathology of prostate tumors was compared with the level of c-cbl expression. The expression level of c-cbl was evaluated visually.

c-cbl was not expressed in surrounding healthy tissue.

For prostate tumors, it was found that:
c-cbl staining was weak in low grade prostate tumors;
c-cbl staining was intense in high grade prostate tumors; and
c-cbl was expressed in epithelial but not in stromal cells.

In BPHs, c-cbl staining was weak.

As prostate cancer is known to be resistant to apoptosis, and as it is known to sustain a very high abnormal expression of IAPs (Krajewska et al. 2003 Clin Cancer Res 9:4914-25), the present results together with the results showing the apoptosis down-regulatory role of c-Cbl demonstrate that c-Cbl is an upstream actor of apoptosis that can alter the apoptotic pathway of cancer, in particular of prostate cancer.

### Example 7

### c-Cbl Up-Regulation is Found in Various Cancers

Immunohistochemistry (IHC) experiments were carried out to evaluate c-cbl expression levels in samples from patients suffering from the following cancers: lung cancer, breast cancer, lymphoma, ovary cancer, brain cancer, colon cancer, thyroid cancer, prostate cancer, melanoma, oesophagus cancer, stomach cancer, liver cancer, kidney cancer, bladder cancer, uterus cancer and pancreas cancer.

For each type of cancer, six different samples were studied, except for colon cancer and melanoma, for which three and one samples were studied respectively. Three of the six samples were obtained from patients in a hospital (CHU Lyon-Sud, Lyon), and the three other samples corresponded to purchased, commercially available slides. For each cancer sample, there was a corresponding control sample of healthy surrounding tissue, except for the melanoma sample.

C-cbl expression was analyzed by immunohistochemistry. The results were analyzed visually. An expression level ranging from “−” (no staining) to ‘+++” (intense staining) was attributed to each sample.

None of the control samples was stained with c-cbl (“−”).

For the cancer samples, c-cbl staining was found to be as shown in table 1 herebelow.

TABLE 1

| Cancer | c-cbl staining |
|---|---|
| lung cancer | ++ to +++ |
| breast cancer | + to ++ |
| lymphoma | ++ to +++ |
| ovary cancer | + to +++ |
| brain cancer | + to ++ |
| colon cancer | + to +++ |
| thyroid cancer | +++ |
| prostate cancer | + to +++ |
| melanoma | +++ |
| oesophagus cancer | — |
| Stomach cancer | — |
| liver cancer | — |
| kidney cancer | — |
| bladder cancer | — |
| uterus cancer | — |
| pancreas cancer | — |

In conclusion, c-cbl is over-expressed in lung cancer, breast cancer, lymphoma, ovary cancer, brain cancer, colon cancer, thyroid cancer, prostate cancer and melanoma. C-cbl could thus be used as a marker for diagnosing these cancers. In addition, c-cbl antagonists are expected to be able to treat these cancers.

Additional experiments were carried out to further investigate the expression level of c-cbl in various tumours, in order to validate the above preliminary results.

More specifically, in situ staining in tissue microarrays assays (TMA) were carried out in sixteen tissues including the following tissues: prostate cancer (prostate adenocarcinoma), breast cancer (infiltrated ductal carcinoma), ovary cancer (serous papillary carcinoma), uterus cancer (squamous cellular carcinoma), brain cancer (astrocytoma), lung cancer (squamous cellular carcinoma), colon cancer (colon adenocarcinoma) and rectal cancer (rectal adenocarcinoma).

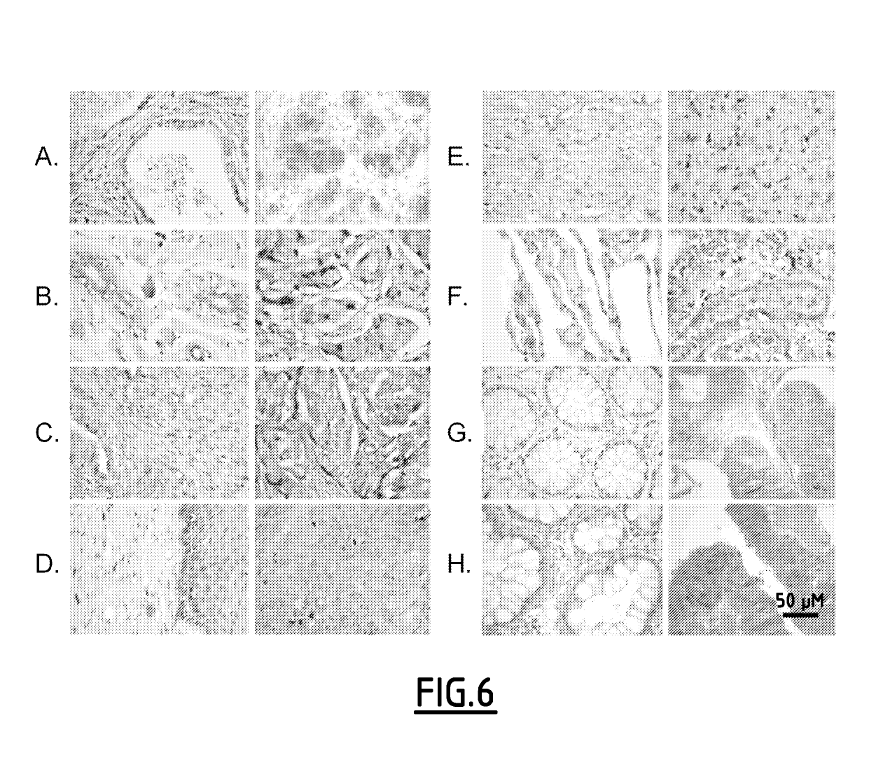

C-cbl over-expression, compared to the corresponding healthy tissue, was detected in seven types of tumours, with different degrees of intensity (FIG. 6). C-Cbl expression compared to normal tissue seemed to be strongly sustained in hormone-dependent tumours (for example in prostate, ovary, uterus and brain cancer). C-cbl was also very highly expressed in rhabdomyosarcoma (cancer of the striated muscle), and in lung, colon and rectal cancers. Some other tumours sustained a quite high c-Cbl staining, but did not significantly differ from control tissues (breast, liver, kidney, bladder, pancreas, lymph nodes, skin, oesophagus and stomach tumours).

The expression level of an endonuclease indicative of oxidative stress was assessed with an anti-APE1/REF1 antibody. It was found that numerous tumours support a high oxidative stress, as shown in table 2 herebelow.

TABLE 2

| | |
|---|---|
| c-Cbl expression | APE1 expression |

| Tissue | NT | T | NT | T |
|---|---|---|---|---|
| Prostate | w | h | w | h |
| breast | h | h | h | h |
| ovary | w | h | h | h |
| uterus | w | h | w | h |
| brain | w | h | w | h |
| muscle | w | h | w | h |
| lung | w | h | h | h |
| liver | h | h | w | h |
| kidney | h | h | h | w |
| bladder | h | h | w | h |
| pancreas | h | h | w | h |
| lymph nodes | h | h | nd | nd |
| colon | w | h | nd | nd |
| rectum | w | h | nd | nd |
| skin | h | h | nd | nd |
| oesophagus | h | h | nd | nd |
| stomach | h | h | nd | nd |

In the above table, “h” stands for high expression, “w” stands for weak expression, “NT” stands for non-tumoral tissue, “T” stands for tumoral tissue and “nd” stands for not determined.

Seven of the eleven tested tissues were positive upon staining with the anti-APE1/REF1 antibody. Among six c-Cbl positive tumours (in which the staining intensity with c-cbl was much stronger than in a healthy tissue), four were also positive for APE1/REF1 only in tumors but not in healthy tissue, namely prostate tumour, uterus tumour, brain tumour and rhabdomyosarcoma. The two other c-Cbl positive tumours, ovary and lung tumours, displayed a high oxidative stress, both in healthy tissues and in tumours. These results suggest that high c-Cbl expression is associated with a strong oxidative stress and, to a lesser extent, with the hormone-dependency of tissues.

Experiments are being carried out in order to correlate the intensity of c-cbl staining with the anatomo-pathologic development of these tumors.

### Example 8

### c-cbl and Reactive-Oxygen Species (ROS)

The above data show that c-Cbl is tightly associated with the survival and/or apoptosis of cells undergoing an oxidative stress. Assuming that tyrosine-kinase receptors are not correctly down-regulated upon strong oxidative stress, it was hypothesized that c-Cbl expression could increase through a positive feedback mechanism. On the other hand, knowing c-Cbl's important contribution to energy expediture, and particularly to fatty acid oxidation, this protein could partly participate to the pathological ROS increase.

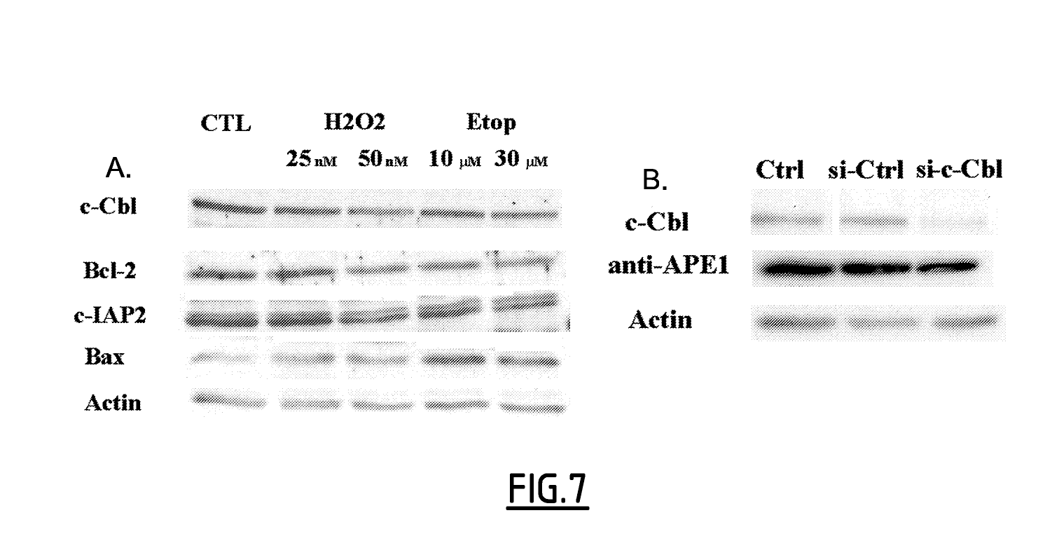

It was found that that c-Cbl is slightly less expressed in LNCaP cell lines cultured in the presence of hydrogen peroxide or etoposide than in the absence of hydrogen peroxide or etoposide (FIG. 7A). The Bcl-2 and c-IAP2 anti-apoptotic factors analyzed here were also slightly less expressed in the presence of hydrogen peroxide or etoposide, whereas the pro-apoptotic Bax protein expression was increased, particularly upon etoposide treatment.

Thus, c-Cbl expression profile is the same as those of other anti-apoptotic, which is perfectly coherent with the fact that c-cbl has an anti-apoptotic affect. In addition, these results show clearly that ROS do not up-regulate c-Cbl.

Conversely, the transient silencing of c-Cbl in LNCaP cell line (FIG. 7B) protects against cellular oxidation since a slight decrease of the anti-APE1/REF1 signal is seen. Therefore, one can deduce that c-Cbl either increases the effects of ROS, or, more probably, plays a role in ROS production.

These data strongly suggest that c-Cbl is involved in ROS production. It is likely that the anti-apoptotic effect of c-Cbl, identified both in vivo and in MEFs, has an effect on survival of tumour cells when they noticeably express c-Cbl. C-cbl is therefore a very attractive target for cancer therapy.

### Example 9

### Apopotic Status of c-cbl Knock-Out (KO) Mice Compared with c-cbl Wild-Type (WT) Mice

In order to investigate the relation between apoptosis and expression of c-Cbl in vivo, the apoptotic status in ventral prostate (VP) of KO mice was compared with the apoptotic status of WT mice.

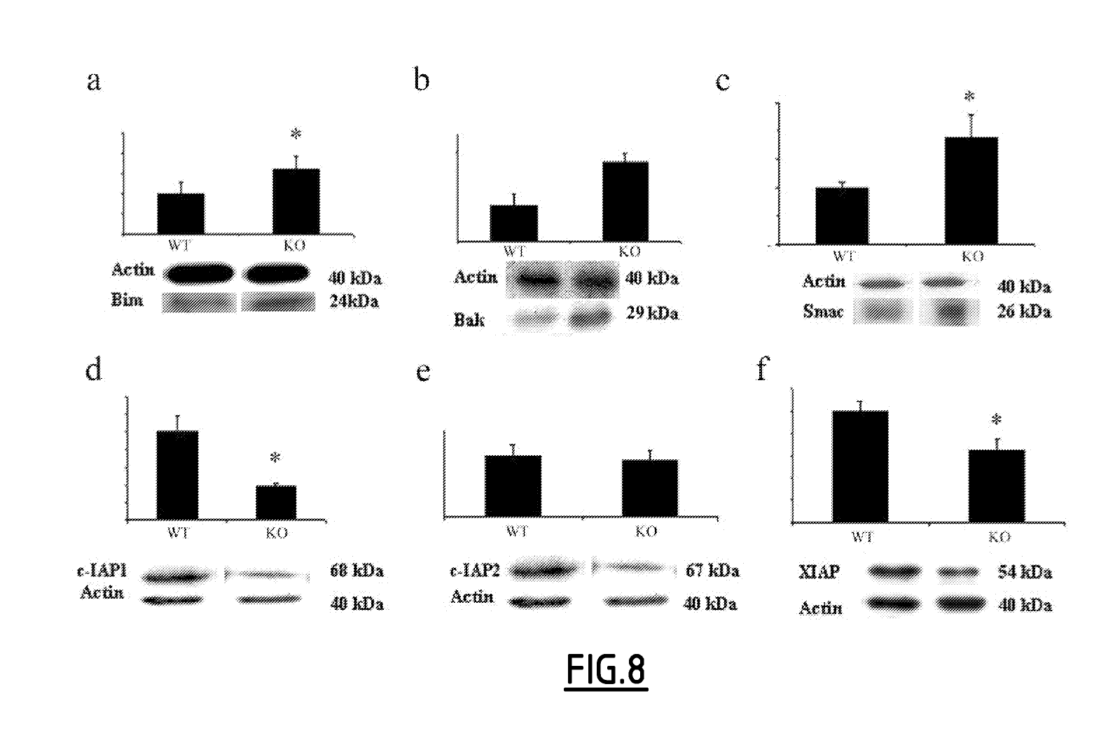

The BH3-Only Bim protein functions by antagonizing anti-survival relatives involved in mitochondrial permeability as Bak or Bax. In VP of KO mice, BimEL is spontaneously more expressed than in VP of WT mice (FIG. 8A). The pro-apoptotic factor Bak was also found to be increased in VP of KO mice (FIG. 8A). The protein Smac/Diablo, released from mitochondria, is a pro-apoptotic IAP-negative regulator that leads to auto-ubiquitination of the IAPs or interferes directly with their caspase-inhibiting domains. The Smac protein is expressed in VP of KO mice at a level that is twice as high as in VP of WT mice (FIG. 8C). Bim over-expression, as well as Bak over-expression, could be the cause of the increased Smac/DIABLO expression.

The expression pattern of IAPs in VP of KO mice versus WT mice was then studied (FIGS. 8D, E and F). C-IAP1, and to a lesser extent XIAP, are spontaneously down-regulated in VP KO, with a one-third decrease for XIAP and a 50% decrease for c-IAP1.

Altogether, these data are consistent with a spontaneous apoptotic/survival unbalance favouring apoptosis in PV of c-Cbl KO mice.

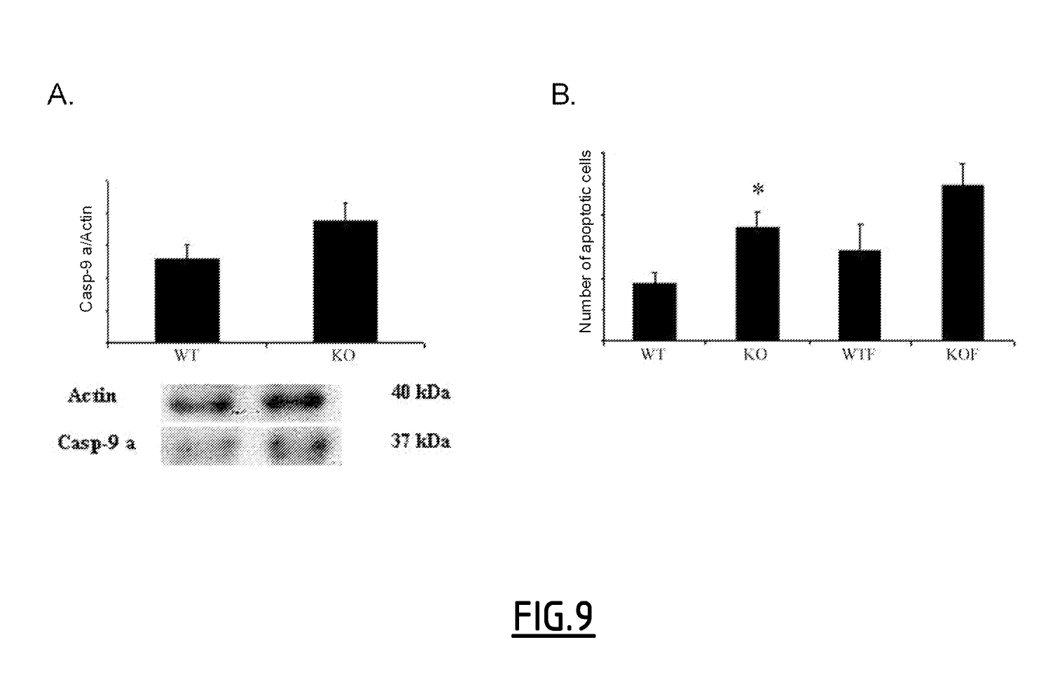

The processed mitochondrial initiator caspase-9 was slightly up regulated in VP of KO mice, this result being coherent with a mitochondrial apoptotic pathway involvement (FIG. 9A).

TUNEL experiments confirmed these data (FIG. 9B), showing a high number of apoptotic cells in c-Cbl KO luminal cells (about 50% higher than in WT luminal cells). Finally, c-Cbl can be considered as an anti-apoptotic regulator in prostate epithelial cells. C-cbl diminishes the physiological apoptotic threshold, and does not particularly interfere with the anti-androgen apoptosis-inducing pattern.

### Example 10

### Discussion of the Results

These experiments focused on the proto-oncoprotein c-Cbl as an actor of the apoptotic process undergone by prostate cells and prostate tumor as well.

The multi-adaptor E3-ubiquitine ligase c-Cbl performs several types of regulation and the potential of c-Cbl for apoptotic regulation had also been suggested in some articles (Sinha et al. 2001 Exp Hematol 29:746-55; Hamilton et al. 2001 J Biol Chem 276:9028-37; Akiyama et al. 2003 Embo J 2003;22:6653-64). El Chami et al. (2005 J Cell Biol 171:651-61) recently showed that c-Cbl has a key role in the regulation of androgen dependent apoptosis of testicular germ cells, which prompted us to investigate such a role in prostate whose maturation and homeostasis depend on androgens. Indeed, the cell death program in prostate is of great significance, since resistance to apoptosis has been demonstrated to be a hallmark of the prostate carcinoma (Krajewska et al. 2003 Clin Cancer Res 9:4914-25; Denmeade et al. 1996 Prostate 28:251-65). Resistance to apoptosis is occurring when the androgen unresponsiveness arises during the course of the disease, as the main event that punctuates prostate carcinoma development, hanging over the vital prognostic of patients (Agus et al. 1999 J Natl Cancer Inst 91:1869-76). The outbreak of the prostate cancer it-self could be affected by the alteration of the apoptotic process yet, as suggested by the up-regulation of the Inhibitors of Apoptosis in PINs (Krajewska et al. 2003 Clin Cancer Res 9:4914-25).

In the frame of the present experiments, it has been found that c-Cbl is essentially and highly expressed in the luminal cells of the ventral prostate (differentiated epithelial cells and not the basal cells). Such a localization and intensity are of great interest for different reasons. First, c-Cbl co-localizes with the Androgen Receptor. It has already been reported that AR is essentially expressed in the luminal cells and it is clear from our experiments that a decrease of in situ testosterone leads to a decrease of c-Cbl expression paralleling AR down expression. The down regulation of AR expression upon testosterone withdrawal has also already been reported. It has also been shown in previous works that androgen down-regulates AR mRNA but up-regulates AR protein half-live. It was finally established that in testis, prostate and seminal vesicles are equally stimulated by androgens and that AR immuno-expression in testis is androgen dependent. In the present study, it was found that the c-Cbl staining strongly decreased as soon as 24 hours of treatment and was confirmed by western blotting that showed the androgen dependency of c-Cbl in prostate. The absence of any alteration of c-Cbl expression in the androgen-independent prostate lobes strengthens the link between c-Cbl and AR. This c-Cbl expression dependency on testosterone demonstrates the probably distinctive and significant regulatory function(s) that this protein performs in this tissue as it does in testis.

Androgens are crucial in driving terminal differentiation of luminal cells and it has been suggested that an androgen-independent transiently amplifying population (TAP) with functional AR may have particular significance in hormone resistant prostate cancer. This population is thought to be androgen responsive through indirect mechanisms and to sustain AR expression by the Keratonocyte Growth Factor (KGF). It will be of great interest to situate c-Cbl in such an intermediate cell population, particularly looking for c-Cbl to escape or not androgen regulation in TAP as well as in tumor cells. The androgen dependency of c-Cbl appeared at the first wave of testosterone showing that it is tightly linked to the growing epithelial cells. Indeed, the level of c-Cbl expression in those cells follows testosterone exposure when compared to a specific marker of epithelial cells (K18).

A second and important aspect attached to the in situ c-Cbl staining described herein is that whatever the time or the dose of the flutamide treatment, no noticeable epithelial disruption occurred. This observation validates the molecular expression data of this work when flutamide treatment is involved. Other works had already proved that such doses were not deleterious and that the number of apoptotic cells reported here or in other works is very low and could hardly account for alteration in c-Cbl expression.

A third and interesting aspect is the increased expression of Bim EL that was constantly observed when c-Cbl is down regulated or invalidated.

MEFs studies showed a drop of IAPs upon apoptotic signals in KO MEFs, and a significant increase of the number of apoptotic KO MEFs upon the same signals. Interestingly, KO MEFs appeared more sensitive to hydrogen peroxide (H₂O₂) at a weak dose (0.1 nM) than WT MEFs, which could be specifically and tightly related to the drastic increase of activated caspase 3 upon H₂O₂. In conclusion MEF c-Cbl is obviously protecting cells against apoptosis particularly induced by a Reactive Oxygen Species (ROS) as Hydrogen Peroxyde.

Cancers are high producer of ROS, particularly the prostate cancer. If ROS amplify the upstream tyrosine phosphorylations or, more likely, dysregulate phosphorylations of RTKs (Khan et al. 2008 FASEB J. 22:910-7), it could be expected from the present experiments that c-Cbl level expression increases to face up to this effect and then interferes with the survival/death balance in favour of survival, in view of the present results obtained with MEFs.

Indeed, all tested prostate tumors displayed a very high c-Cbl expression, which could be the cause of the IAP increase expression and then the strong resistance to apoptosis. Interestingly, this mechanism could appear as soon as the prostate cancer outbreaks as the IAPs expression alterations were observed in PINs.

Our results showed also that other cancer than prostate were concerned with the increase c-Cbl expression and then extend the interest given to this tumoral marker and point out the shared identity of the mechanisms of this tumoral alteration.

### Example 11

### Summary of the Results

Here it has been shown that c-Cbl is highly expressed in epithelial cells of ventral prostate in an androgen dependent manner. It has also been found that c-Cbl is anti-apoptotic. Particularly, its invalidation in MEFs drastically reduced the expression of Inhibitors of Apoptosis (IAPs). An abnormally high expression of c-Cbl was found in human tumors, which are known to be resistant to apoptosis and over-express IAPs, as does the intraepithelial neoplasia (PIN). c-Cbl, which is highly expressed in epithelial ventral prostate cells, is a probable down-regulator of apoptosis in mice and rats and undoubtelly in primary MEFs. These findings strongly suggest that c-Cbl is involved in the abrogation of apoptosis in human tumor and that c-Cbl is responsible for the resistance to H₂O₂-inducible apoptosis. c-Cbl can also be considered as a tumor marker.

It has been found that the expression of c-cbl lied spontaneously in the differentiated epithelial cells of the ventral lobe of the rat. The androgen-dependence of c-Cbl expression manifests itself only in the epithelial cells of this lobe, the development and maintenance of which are known to depend on androgens. These results were obtained by means of experiments carried out after administration of the androgen antagonist flutamide, or else by castration and replacement administration of testosterone. It was also shown that the c-Cbl expression level increased significantly with prostate development in mice (15 to 20 days post-natal).

The apoptosis induced by the administration of flutamide was found to be associated with a negative regulation of the c-Cbl expression level in the ventral prostate of the rat. It was further shown that in the rat, with administration of flutamide, expression of the inhibitor of apoptosis c-IAP2 was significantly decreased, whereas the pro-apoptotic factor Bim EL was gradually overexpressed.

In order to be sure that the regulation exerted by c-Cbl in the prostate cells can be observed in another system, MEF KO and MEF WT were established and their sensitivity to apoptosis was tested in the presence of H₂O₂ and of etoposide. It was shown that the level of expression of the inhibitor of apoptosis c-IAP2 was significantly decreased when the cells were placed in the presence of one or other of these apoptosis inducers. The number of MEF KO undergoing apoptosis under the effect of these same inducers was much higher when the cells were placed in the presence of H₂O₂ (DAPI experiment). Thus, while the spontaneous apoptosis of these constantly proliferating cells is very low, the absence of c-Cbl renders these cells very sensitive to hydrogen peroxide. These results are in agreement with those obtained in vivo for the prostate: c-Cbl appears to contribute to the resistance to apoptosis, especially, it would appear, when there are redox potential alterations. It is interesting to note that prostate cancers, particularly when they are aggressive, are described as exhibiting a strong alteration of the redox potential.

It has further been shown by western blotting that hormone-dependent prostate cancers exhibit a very high spontaneous expression of c-cbl compared with the surrounding healthy tissue. These experiments suggest that the intensity of c-Cbl expression follows the anatomopathological severity of the tumor. Other tumors were tested by immunohistochemistry, and it was found that c-cbl is overexpressed in lung cancer, breast cancer, lymphoma, ovary cancer, brain cancer, colon cancer, thyroid cancer, prostate cancer and melanoma as well.

In summary, the experiments concern the demonstration, by means of immunohistochemical or western blotting experiments, of the spontaneous overexpression of the c-cbl proto-onco protein in human prostate adenocarcinomas. It also concerns the possibility of activating the programmed cell death of the same tumor cells by means of treatments using c-cbl RNA interference techniques. The results imply that the expression of c-cbl is very high due to the oxidative stress present in prostate cancers, thus leading to a resistance of these tumor cells to apoptosis. They also imply that the same mechanism is involved in increased c-Cbl expression in other tumor cells.

It has further been shown that mice that are knocked-out for c-cbl present an apoptosis rate that is spontaneously higher than mice that are wild-type for c-cbl. This result further confirms the anti-apoptotic effect of c-cbl.

Experiments carried out with LNCaP cell lines led to the following conclusions:
like the Bcl-2 and IAP anti-apoptotic factors, c-cbl expression is reduced in the presence of H₂O₂ and etoposide. C-cbl has thus the typical expression profile of an anti-apoptotic factor; and
the reduced expression of c-cbl seen in the presence of H₂O₂ is not due to H₂O₂, but to the contrary, it appears that a reduced expression of c-cbl would lead to a decrease in ROS like H₂O₂.

### Example 12

### c-cbl RNA Interference Experiments

c-cbl RNA interference experiments are carried out on human tumor cells, either obtained from patients or from divers cancerous cell lines in addition to LNCaP (e.g. DU145 or PC3) with H₂O₂ apoptosis activation.

c-cbl RNA interference experiments are also carried out on mice models such as the TRAMP mouse model and the CWR22Rv1 mouse model. In the TRAMP mouse model, p53 is inactive due to the presence of the T antigen. In the CWR22Rv1 mouse model, it is possible to obtain human prostate tumor xenografts which have derived so as to become androgen-independent, although the mice have the androgen receptor. The expression level of c-cbl is measured and correlated with the tumor grade, apoptosis level, and/or expression level of Sprouty 2, IAPs (in particular XIAP), Bim, Smac/DIABLO, AIF and AR. In addition, apoptosis may be induced in these tumor cells and/or mice models. Moreover, siRNA c-Cbl can be injected intra-peritonealy to those mice and reduction of the tumors is measured.

1-18. (canceled)

19. A method for treating or preventing a cancer associated with resistance to apoptosis, which comprises administering a c-cbl antagonist to a subject in need thereof.

20. The method of claim 19, wherein said cancer is selected from the group consisting of lung cancer, prostate cancer, breast cancer, ovary cancer, brain cancer, colon cancer, colorectal cancer, thyroid cancer, uterus cancer, rectal cancer, cancer of the striated muscle, testicular cancer, lymphoma and melanoma.

21. The method of claim 19, wherein said antagonist is a small molecule or a nucleic acid selected from the group consisting of an interfering RNA, an antisense DNA and an aptamer.

22. The method of claim 21, wherein said antagonist is an interfering RNA comprising SEQ ID NO: 2 and SEQ ID NO: 3.

23. The method of claim 19, wherein said treatment is a treatment by combination chemotherapy.

24. The method of claim 19, wherein said c-cbl antagonist is administered in combination with an effective amount of at least one compound selected from the group consisting of an alkylating agent, an antimetabolite, an antimitotic, a topoisomerase inhibitor, a hormonal therapy drug, a signaling inhibitor, an aromatase inhibitor, a differentiating agent, a monoclonal antibody, a biologic response modifier and an antiangiogenic agent.

25. An in vitro method of screening for drugs for the treatment of a cancer associated. with resistance to apoptosis comprising the steps of:
a) providing a test compound; and
b) determining whether said test compound inhibits c-cbl;
wherein the determination that said test compound inhibits c-cbl indicates that said test compound is a drug for the treatment or the prevention of a cancer associated with resistance to apoptosis.

26. The method of claim 25, wherein said cancer is selected from the group consisting of lung cancer, prostate cancer, breast cancer, ovary cancer, brain cancer, colon cancer, colorectal cancer, thyroid cancer, uterus cancer, rectal cancer, cancer of the striated muscle, testicular cancer, lymphoma and melanoma.

27. The method of claim 25, wherein said test compound is a small molecule or a nucleic acid selected from the group consisting of an interfering RNA, an aptamer and an antisense DNA.

28. An in vitro method of diagnosing a cancer comprising the steps of:
a) providing a biological sample from a patient susceptible of suffering from cancer;
b) determining c-cbl expression level in said biological sample; and
c) comparing the c-cbl expression level measured at step (b) with a value or a range of values measured in an unaffected biological sample;
wherein the determination that the c-cbl expression level measured at step (b) is higher than the value or the range of values measured in the unaffected biological sample indicates that said patient suffers from cancer.

29. An in vitro method of diagnosing the aggressiveness of a cancer comprising the steps of:
a) providing a biological sample from a patient susceptible of suffering from cancer;
b) determining c-cbl expression level in said biological sample; and
c) comparing the c-cbl expression level measured at step (b) with values or ranges of values measured in biological samples from:
individuals suffering from a non-aggressive cancer; and
individuals suffering from an aggressive cancer;
wherein:
the determination that the c-cbl expression level measured at step (b) is identical to the value or falls within the range of values measured in biological samples from individuals suffering from a non-aggressive cancer indicates that said cancer is not aggressive; and
the determination that the c-cbl expression level measured at step (b) is identical to the value or falls within the range of values measured in biological samples from individuals suffering from an aggressive cancer indicates that said cancer is aggressive.

30. The method of claim 28, wherein said cancer is selected from the group consisting of lung cancer, prostate cancer, ovary cancer, uterus cancer, brain cancer, colon cancer, colorectal cancer, rectal cancer and cancer of the striated muscle.

31. The method of claim 30, wherein said cancer is prostate cancer, and wherein said biological sample from a patient susceptible of suffering from cancer comprises prostate epithelial cells and/or prostate differentiated luminal cells.

32. An in vitro method for selecting a patient suffering of a cancer suitable to be treated by an aggressive chemotherapy comprising the step of determining c-cbl expression level in a biological sample from said patient, and selecting the patient having a high expression level of c-cbl.

33. The method of claim 32, wherein said aggressive chemotherapy is a combination chemotherapy carried out with high doses of drugs.

34. The method of claim 32, wherein said aggressive chemotherapy comprises the administration of a c-cbl antagonist.

35. An in vitro method for selecting a patient suffering of a cancer suitable to be treated by a c-cbl antagonist comprising the step of determining c-cbl expression level in a biological sample from said patient, and selecting the patient having a high expression level of c-cbl.

36. A method of treating or preventing a cancer associated with resistance to apoptosis comprising the steps of:
a) determining c-cbl expression level in a biological sample from said patient;
b) selecting the patient having a high expression level of c-cbl; and
c) administering an effective amount of a c-chl antagonist to said patient having a high expression level of c-cbl.
